(19) United States
(12) Patent Application Publication
(10) Pub. No.: US 2011/0311505 A1
(43) Pub. Date: Dec. 22, 2011

(54) Photosensitive Aminoacid-Monomer Linkage and Bioconjugation Applications in Life Sciences and Biotechnology

(75) Inventors: Arzu Ersoz, Eskisehir (TR); Deniz Hur, Eskisehir (TR); Filiz Yilmaz, Eskisehir (TR); Adil Denizli, Eskisehir (TR); Ayca Atilir Ozcan, Eskisehir (TR); Sibel Emir Diltemiz, Eskisehir (TR); Suzan Yazar, Eskisehir (TR); Ozlem Bicen, Eskisehir (TR); Sibel Buyuktiryaki, Eskisehir (TR); Rustem Kecili, Eskisehir (TR); Guner Saka, Eskisehir (TR); Tugba Findik, Eskisehir (TR)

(73) Assignee: Ridvan Say, Eskisehir (TR)

(21) Appl. No.: 13/203,833

(22) PCT Filed: Dec. 11, 2009
(86) PCT No.: PCT/IB2009/055707

§ 371 Date: Aug. 29, 2011

(51) Int. Cl. A61K 38/44; B05D 7/00; G01N 33/566; C12Q 1/34; C12Q 1/44; C12Q 1/26; C12Q 1/28; C12Q 1/02; C12N 9/96; G01N 21/75; C08J 3/28; C07K 1/107; C07D 209/20; C07C 235/78; C07C 323/59; A61P 35/00; C07F 17/00; B05D 5/06; B82Y 40/00; B82Y 5/00; B82Y 15/00

(52) U.S. Cl. 424/94.3; 427/157; 427/214; 435/7.23; 435/18; 435/19; 435/25; 435/28; 435/29; 435/188; 436/501; 436/86; 522/87; 530/364; 530/380; 530/391.1; 530/391.3; 530/400; 530/408; 530/409; 548/496; 562/445; 562/556; 204/157.75; 204/157.71; 977/746; 977/773; 977/847; 977/896; 977/915; 977/920; 977/810

(57) ABSTRACT

This invention is related to preparation of photosensitive ruthenium based aminoacid monomers and oligomers, aminoacid monomer-protein cross-linking using photo sensitat ion and conjugation on micro and nano-structures by ruthenium-chelate based monomers. Its vast range biotechnolgy applications of multifunctional, biocompatible, stabilE and specific micro and nanobio-conjugates, which will stand-alone or simultaneously enable (i) both purification and determination, (ii) both targeting and imaging and theranostics and (iii) catalysis and determination. The construction and method of preparation is applicable to silica materials, superparamagnetic particles, QDs, CNTs, Ag/Au nanoparticles and Au surfaces and polymeric materials. The photosensitive aminoacid monomer linkers can react via chemically and biocompatible to a lot of different micro and nano-surface and then to the protein when they act as a single-step cross-linking reaction using irradiation. The photosensitive conjugation based on click biochemistry can be carried out at mild conditions, independent of pH and temperature, without affecting conformation and function of protein.

### FIELD OF THE INVENTION

This invention is related to preparation of photosensitive ruthenium based aminoacid monomers and oligomers, protein conjugating method and their use in life sciences and biotechnology.

### PRIOR ART

In the field of life sciences and biotechnology, nanobioconjugates can make a worthy contribution to the development of new diagnostic and delivery systems. That is to say that, they offer optical, magnetic, biocompatible, biostabil and orientation properties, for sensitive detection, drug targeting and therapy, isolation and purification plus biocatalysis. A major step toward the applicability of the nanobioconjugates for sensorics, drugs, separators and catalysators is therefore the design and enrichment of adequate conjugation of their nanoparticle surface. This conjugation step should lead to a chemical and physical stabilization of the nanostructures as well as the ability.

The most commonly used conjugation chemistry for protein and peptide immobilization is covalent attachment via primary amino groups onto a carboxy-modified nanosurface following activation with N-ethyl-N′-dimethylaminopropyl-carbodiimide (EDC) and N-hydroxysuccinimide (NHS). However, while applicable for many proteins and peptides, these very acidic isoelectric points (pI<4.0) will not be electrostatically concentrated into the matrix which is essential for effective covalent immobilization (Catimet B., et.al., J. Biochem. Biophys. Methods, 49, 2001, 289). Immobilization can be defined as the attachment of biomolecules to a surface resulting in reduction or loss of mobility. In some cases, immobilization may lead to partial or complete loss of protein activity, due to random orientation and structural deformation. Oriented immobilization of active proteins are good steric accessibilities of active binding sites and increased stability. Protein inactivation starts with the unfolding of the protein molecule through the contact of water with hydrophobic clusters located on the surface of protein molecules. Thus, the reduction of the nonpolar surface area by the formation of a suitable biospecific complex of Protein A or by use of carbohydrate moieties may stabilize proteins (Turkova, J., J. Chromatog. B, 722, 1999, 11). Antibodies have been used in the design of separation and biosensors materials for decades due to their extremely high specificity. It is worth nothing that, in the binding processes, the antibody needs to be attached to a solid matrix, and the antibody immobilization may be in many cases a critical step in the design of separation and biosensor materials. The first requirement of the system requires to have a final inert surface that prevents the unspecific adsorption on the matrix of other components of the samples or of the molecules used to report the signal. If this is not exercised, the specificity of the systems may be greatly reduced. Another important feature that the immobilization systems should accomplish is to maintain the antibody functionality. That is to say that, it should not produce severe distortions of the antibody structure. However, in some instances, the immobilisation of antibodies presents a further complication. Infact, if the analyte is a protein or a cell, only antibodies properly oriented regarding the surface of the support will be able to recognize it. For steric reasons, antibodies oriented toward the support surface can not bind antigen. The balancing of both features will produce the final synergie between producebility and sensitivity of the biosensor and bioseparation process (Batalla, P. Et. Al., Biomacromolecules, 2008,9, 719). On the other hand, polyvalent interactions are characterized by the simultaneous binding of multiple ligands on a biomolecule or a nano-surface to multiple receptors. Polyvalent interactions can be collectively much stronger than corresponding monovalent interactions. They can furthermore provide the basis for mechanisms of both agonizing and antagonizing biological interactions that are fundamentally different from those available in monovalent systems (Whitesides, et.al., Angew. Chem. Int. Ed., 37, 1998, 2754).

Photoimmobilization demands the presence of mediating photosensitive reagents, generally activated by incident light of an appropriate wavelength. After light activation, the reagents undergo distinct chemical processes that finally lead to the formation of covalent bonds between the photogenerated intermediates and the biomolecules (Rusmini, F. et.al., Biomacromolecules, 8, 2007, 1775). The underlying chemistry is proposed in the literature to involve the formation of radicals allowing tyrosine residues to give covalent bonds with another tyrosine and to use a strategy to oxidative protein-protein crosslinking using photosensitization (Fancy, D. A., and Kodadek, T. Proc. Natl. Acad. Sci. USA, 96, 1999, 6020; Brown, K. C., and Kodadek, T., Met. Ion. Biol. Sys., 38, 2001, 351; Duroux-Richard, I. et al., Chem. & Biol. 12, 2005, 15).

Certain nanomaterials are ideal probe candidates because of their (i) small size (1-100 nm) and correspondingly large surface-to-volume ratio, (ii) chemically tailorable physical properties, (iii) unusual target binding properties, and (iv) overall structural robustness. Tailorable physical properties and oriented surface modification are very important aspects of nanomaterials. Indeed, in this regard, nanomaterials and biology have a sustained history as nanoparticles have been used bio-conjugation and cellular labeling agents for the past four decades (Rosi, N. L., Mirkin, C. A., Chem. Rew., 105, 2005, 1547). At this point in time, the use of nanobio-conjugates for life sciences and biotechnology applications is one of the fastest moving fields of nanobiotechnology. To apply nanoparticles in biological systems or aqueous environment it is essential to modulate the chemical nature of nanoparticle surfaces to alter their biocompatibility and add additional biochemical functionalities and stabilities. By employing different conjugation technologies they can not only be rendered biocompatible, but also, to fulfill tasks. These embody receptor targeting, sensing, imaging, catalysis or preconcentrator. To achieve this goal different monomeric or polymeric coatings are applied to provide biocompatibility and additional bioconjugation (also for multivalent interaction) for targeting those particular drugs which prevent the disease spreeding and other applications (Hezinger A. F. E., Tessmar J., and Gopferich, A., Eur. J. Pharm. Biopharm., 68, 2008, 132).

WO03065888 relates to novel tumor specific phototherapeutic and photodiagnostic agents. The compounds consist of a carbocyanine dye for visualization, photosensitizer for photodynamic treatment, and tumor receptor-avid peptide for site-specific delivery of the probe and phototoxic agent to diseased tissues.

WO0012575 relates to a method for synthesizing continuous, polymeric solid phase supporting materials with spatially defined and interspaced reaction sites. The solid phase supporting material is comprised of a supporting matrix and graft copolymer chains with reactive groups. The surface of the supporting matrix is coated with a photoinitiator. The supporting matrix and the photoinitiator are exposed in the presence of an unsaturated functional monomer.

### BRIEF DESCRIPTION OF INVENTION

The objective of this invention is to preparation of photosensitive ruthenium based aminoacid monomers and oligomers, aminoacid monomer-protein cross-linking using photosensitation and conjugation approach on micro and nano-structures by ruthenium-chelate based monomers. Its vast range applications of multifunctional, biocompatible, stabil and specific micro and nanobio-conjugates, which will stand-alone or simultaneously enable ; both purification and determination, both targeting and imaging and theranostics and catalysis and determination. The construction and method of preparation is applicable to silica materials, superparamagnetic particles, QDs, CNTs, Ag/Au nanoparticles and Au surfaces and polymeric materials. The photosensitive aminoacid monomer linkers can react via chemically and biocompatible to a lot of different micro and nano-surface and then to the protein when they act as a single-step cross-linking reaction using irradiation. The conjugation based on click biochemistry can be carried out at mild conditions, independent of pH and temperature, without affecting conformation and function of protein.

### DETAILED DESCRIPTION OF INVENTION

“Photosensitive ammoacid-monomer linkage and bioconjugation applications” realized to fulfill the objective of the present invention is illustrated in the accompanying figures, in which,

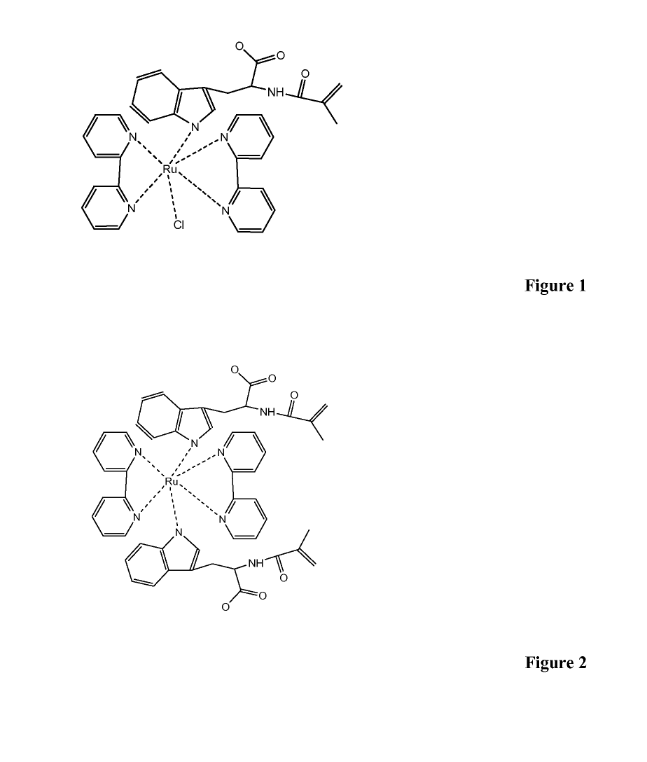

FIG. 1 shows tyrptophan aminoacid monomer having Chlorobis(2-2′-bipyridyl) MATrp-ruthenium(II) photosensitive chelate (preferably, the monomer linker moiety is selected from the methacryloyl)

FIG. 2 shows two tyrptophan amino acid monomers having Bis(2-2′-bipyridyl)bis(MATrp)-ruthenium(H) photosensitive chelate (preferably, the monomer linker moiety is selected from the methacryloyl)

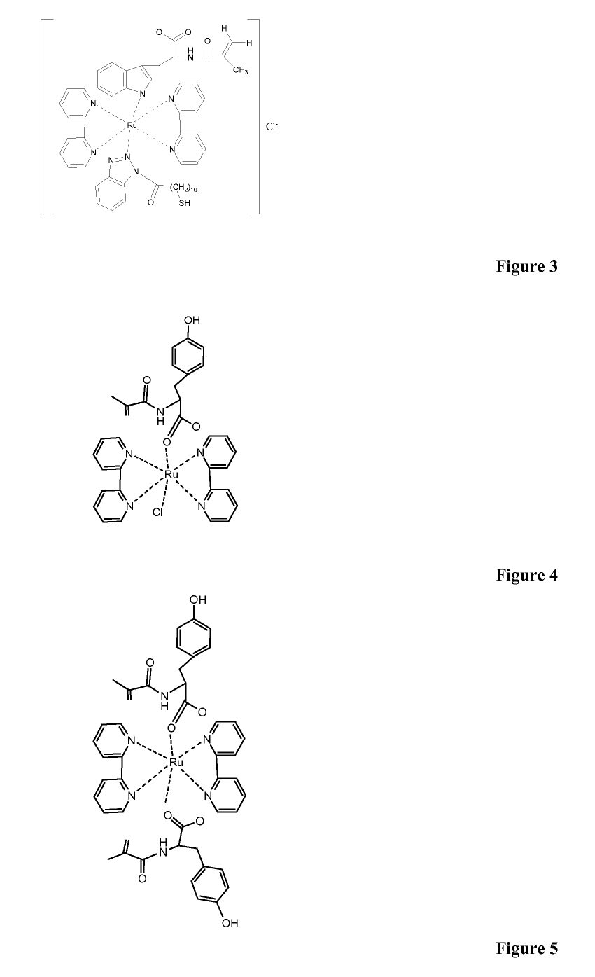

FIG. 3 shows tyrptophane aminoacid monomer and MUABt ligands having Bis(2-2′-bipyridyl)-MATrp-MUABt ruthenium(H) photosensitive chelate (preferably, the monomer linker moiety is selected from the methacryloyl)

FIG. 4 shows tyrosine aminoacid monomer having Chlorobis(2-2′-bipyridyl)MATyr-ruthenium(II) photosensitive chelate (preferably, the monomer linker moiety is selected from the methacryloyl)

FIG. 5 shows two tyrosine aminoacid monomer having Bis(2-2′-bipyridyl)bis(MATyr)-ruthenium(II)photosensitive chelate (preferably, the monomer linker moiety is selected from the methacryloyl)

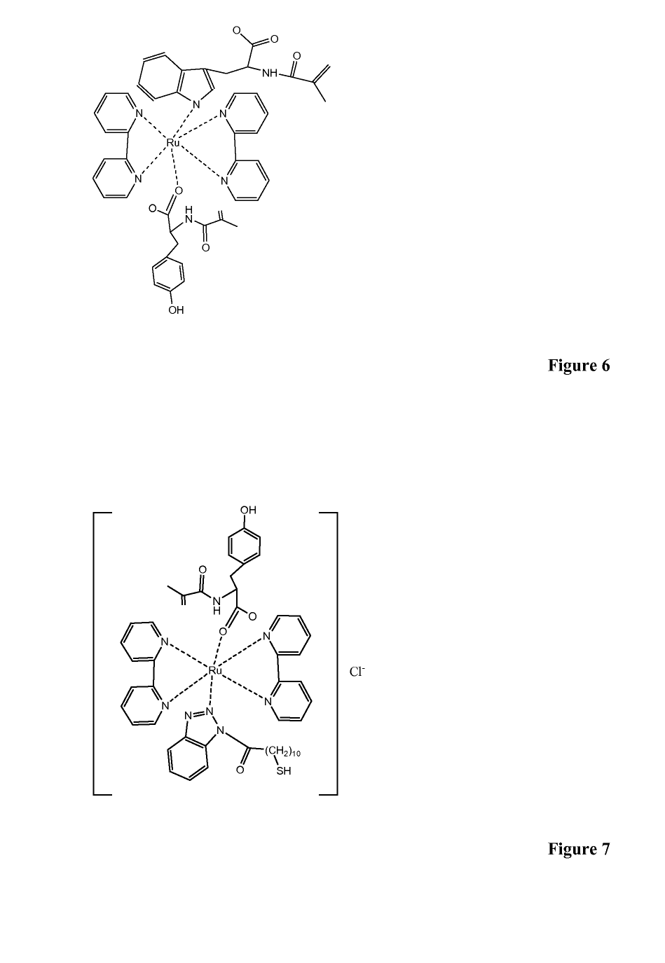

FIG. 6 shows tyrosine and tryptophan aminoacid monomers having bis(2-2′-bipyridyl)MATyrMATrp-ruthenium(II)photosensitive chelate (preferably, the monomer linker moiety is selected from the methacryloyl)

FIG. 7 shows tyrosine aminoacid monomer and MUABt having Bis(2-2′-bipyridyl)MATyrMUABt-ruthenium(H)photosensitive chelate (preferably, the monomer linker moiety is selected from the methacryloyl)

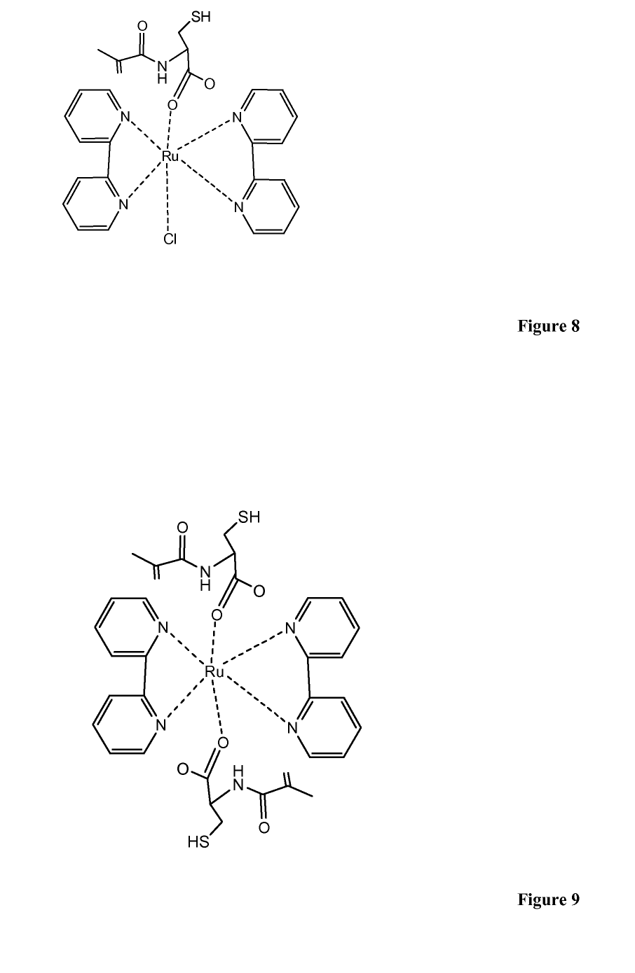

FIG. 8 shows cysteine aminoacid monomer having Chlorobis(2-2′-bipyridyl) MACys-ruthenium(H) photosensitive chelate (preferably, the monomer linker moiety is selected from the methacryloyl)

FIG. 9 shows two cysteine aminoacid monomers having bis(2-2′-bipyridyl)bis(MACys) ruthenium(H) photosensitive chelate (preferably, the monomer linker moiety is selected from the methacryloyl)

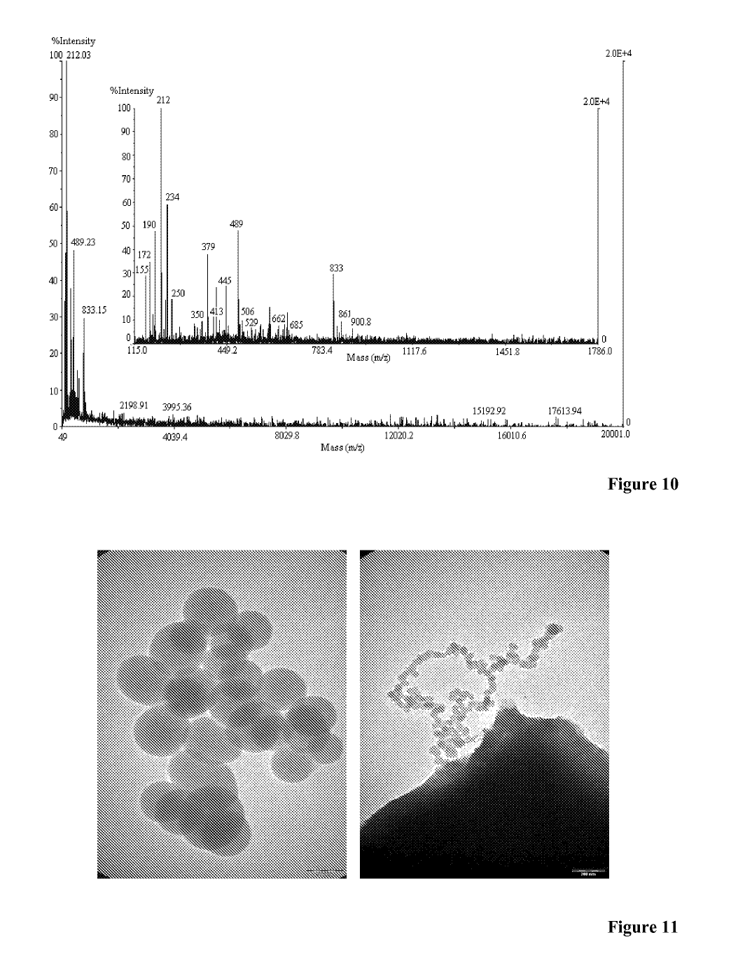

FIG. 10 shows MALDI-TOF/MS spectrum of ([Ru (bpy)₂MATrp-MATyr]ₙ) oligomer.

FIG. 11 shows transmission emission microscope (TEM) images of Nano BSA particles according to an example of the invention.

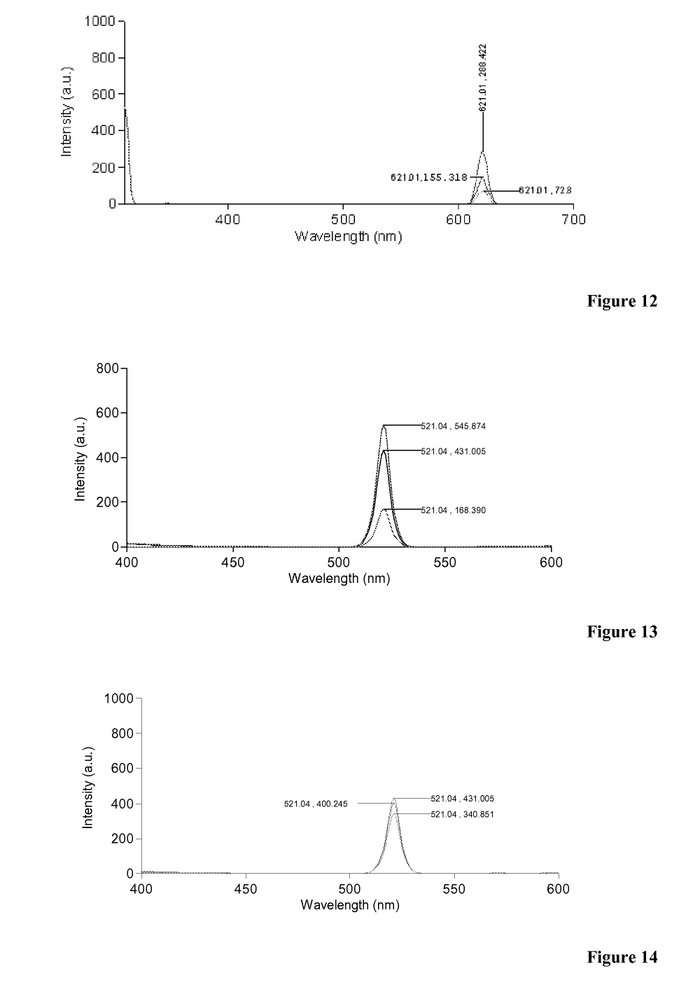

FIG. 12 shows comparison of photoluminescence spectrum of nano BSA, HRP conjugated BSA and HRP conjugated BSA with DAB according to this example of the invention.

FIG. 13 shows photoluminescence spectrum of nanotransferrin and antitransferrin affinity interaction according to an example of the invention.

FIG. 14 shows fluorescence spectroscopy analyses of 4, 8 and 16 ppm antitransferrin solution according to this example of the invention.

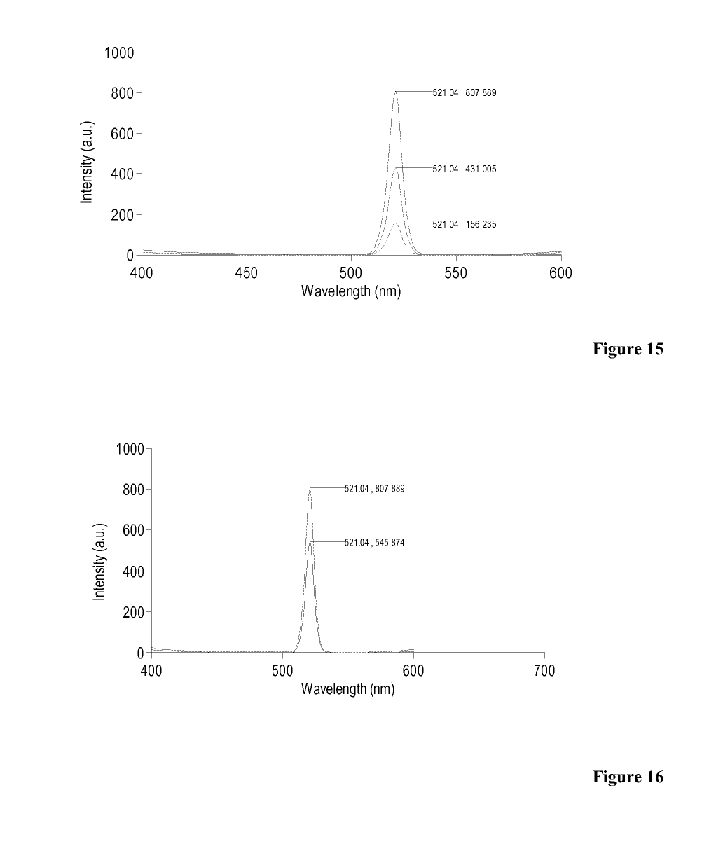

FIG. 15 shows fluorescence spectroscopy analysis of 16 ppm transferrin solution, 16 ppm antitransferrin solution and their mixture according to this example of the invention.

FIG. 16 shows Fluorescence spectroscopy analysis of transferrin-antitransferrin mixture (above peak) and nanotransferrin-antitransferrin mixture (below peak) according to this example of the invention.

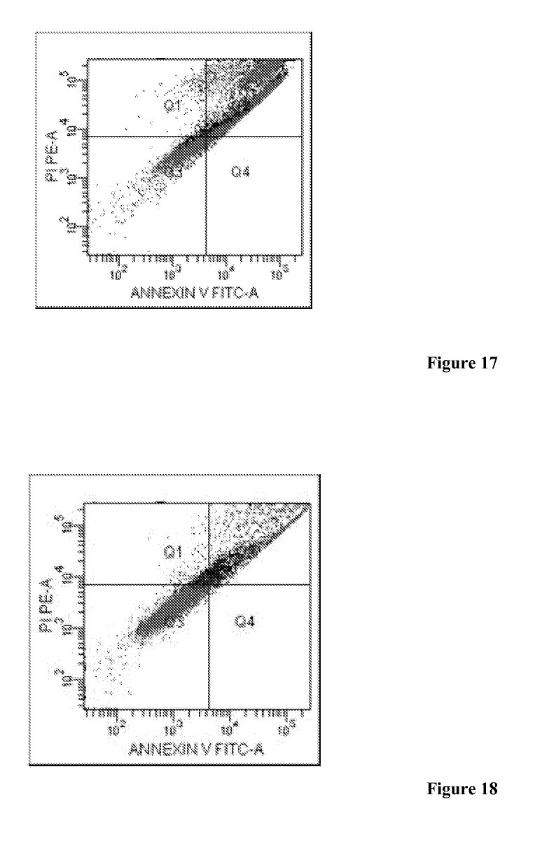

FIG. 17 shows flow cytometry analyses of methotrexate conjugated nanotransferrin for killing the cancer cells according to an example of the invention.

FIG. 18 shows flow cytometry analyses of nanoTNF-α for killing the cancer cells. Q1 ratio (necrotic cells) was 8.5%, Q3 ratio (viable cells) was 69.8% and Q2 ratio (late apoptotic cells) and Q4 ratio (early apoptotic cells) were respectively 21.0% and 0.6%.

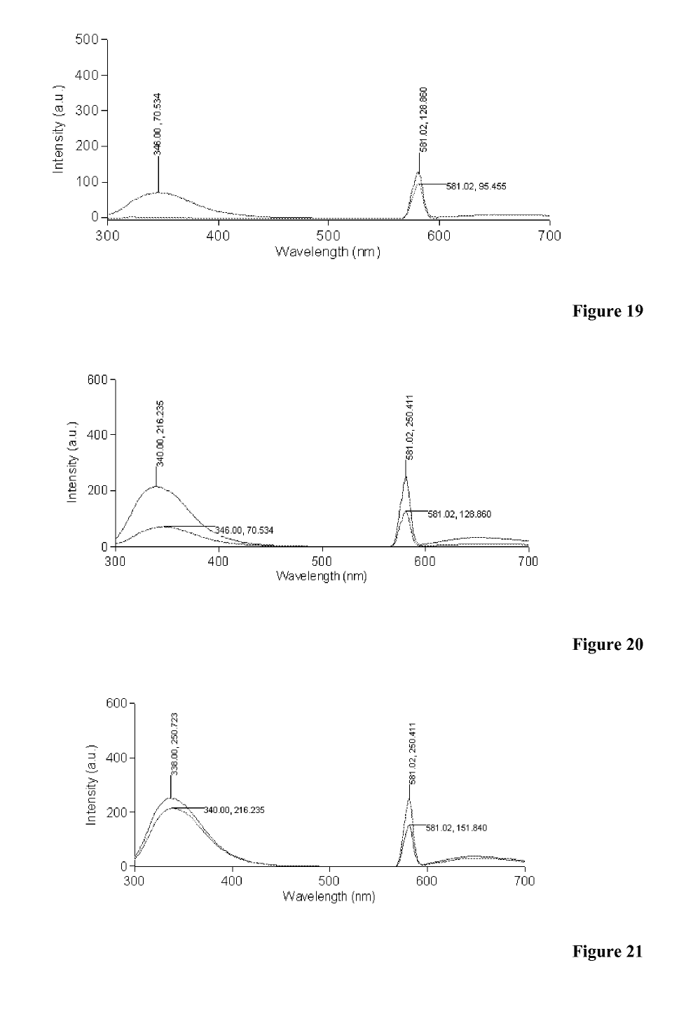

FIG. 19 shows comparison of photoluminescence spectrum of Gold-Protein A interaction with Nano IgG for staphylococcal targeting.

FIG. 20 shows comparison of photoluminescence spectrum of Anti IgG Antibody Interaction with Nano IgG conjugated Gold-Protein A.

FIG. 21 shows comparison of photoluminescence spectrum of Anti IgM Antibody Interaction With [Anti IgG-Nano IgG] Conjugated Gold-protein A.

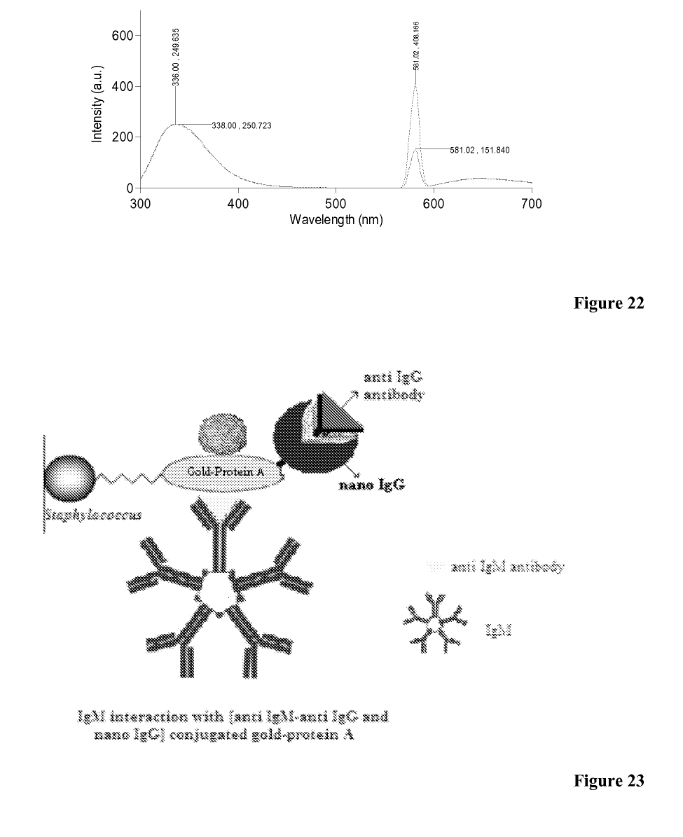

FIG. 22 shows comparison of photoluminescence spectrum of IgM Interaction With Anti IgM-conjugated onto [Anti IgG-Nano IgG] Conjugated Gold-protein A.

FIG. 23 shows Schematic representation to IgM interaction with anti IgM antibody conjugated onto [anti IgG antibody-nano IgG] conjugated gold-protein A.

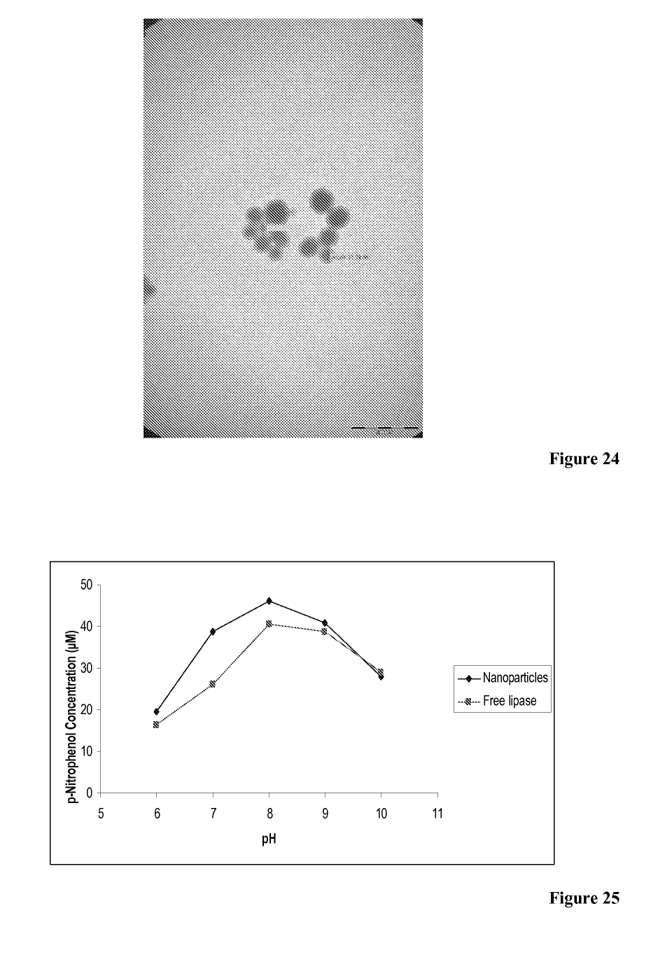

FIG. 24 shows transmission emission microscope (TEM) image of Nano lipase particles according to an example of the invention.

FIG. 25 shows Effect of pH on p-NPP hydrolysis using lipase and nanoparticles.

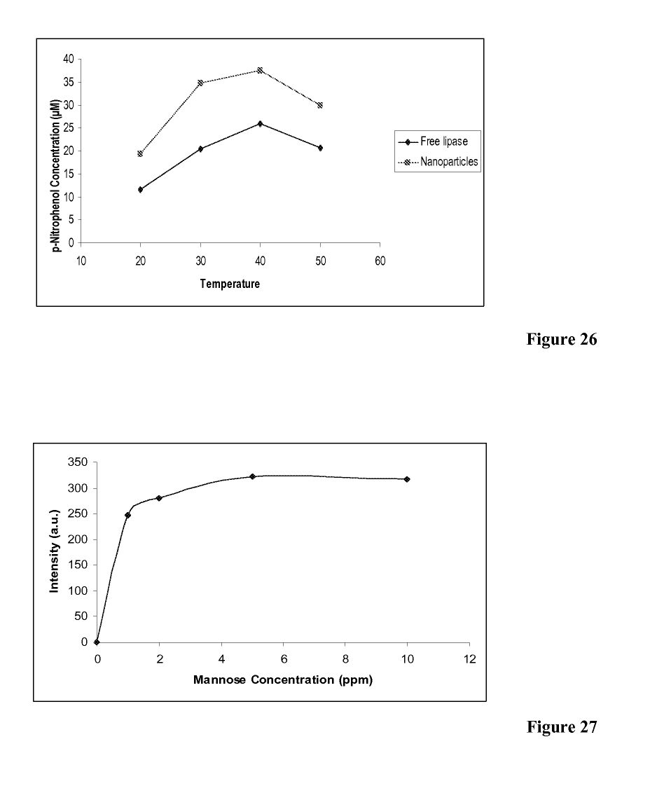

FIG. 26 shows effect of temperature on p-NPP hydrolysis using lipase and nanoparticles.

FIG. 27 shows Effect of mannose concentration on mannose adsorption.

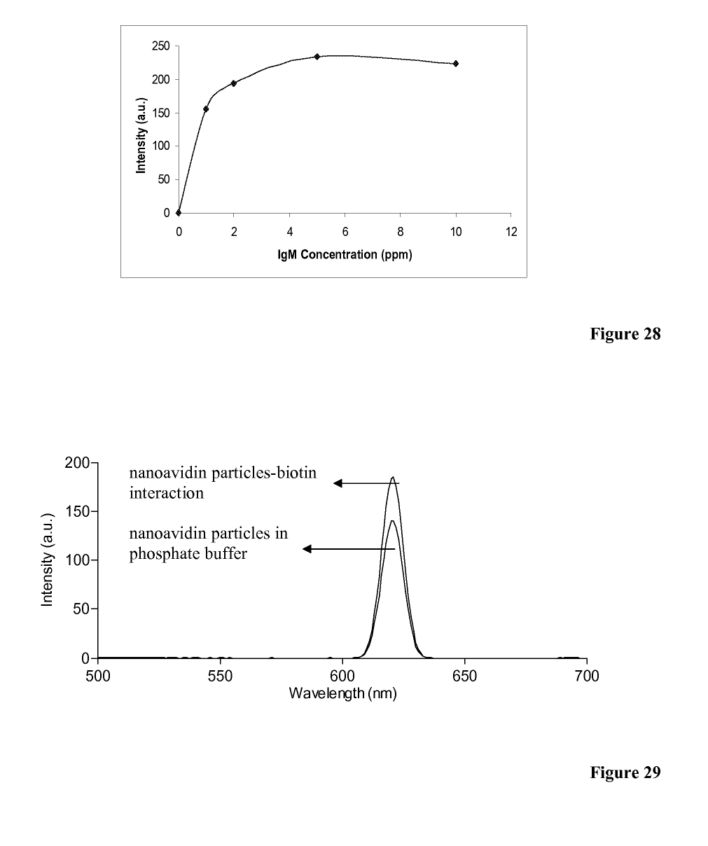

FIG. 28 shows Effect of IgM concentration on IgM adsorption.

FIG. 29 shows comparison of fluorescence changing of nanoavidin nanoparticles interaction with biotin at 620 nm for 20° C.

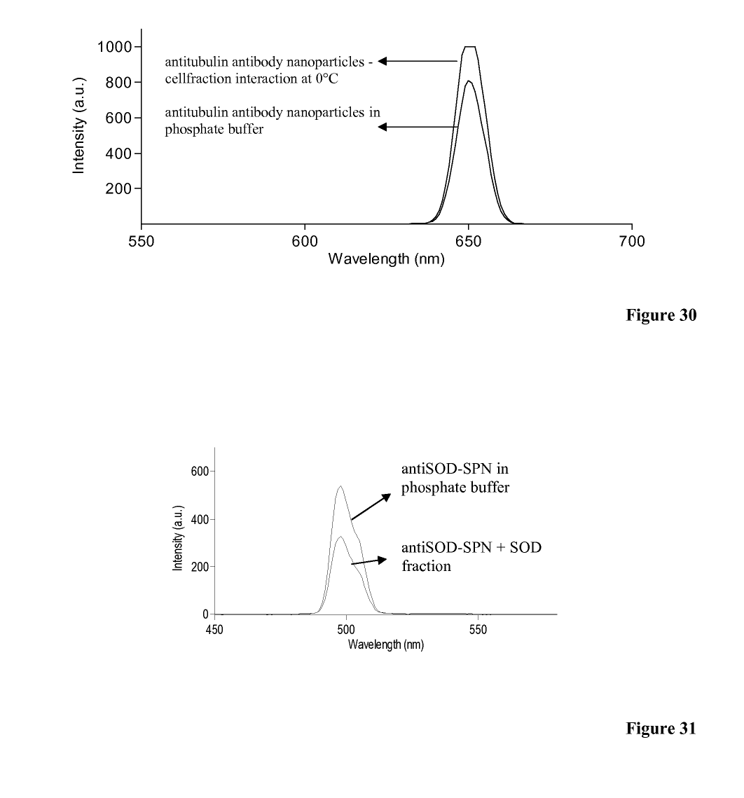

FIG. 30 shows comparison of fluorescence changing of antitubulin antibody nanoparticles interaction with cell fraction at 650 nm for 020 C.

FIG. 31 shows the quenching of emission by interaction of antiSOD-SPN and SOD fraction.

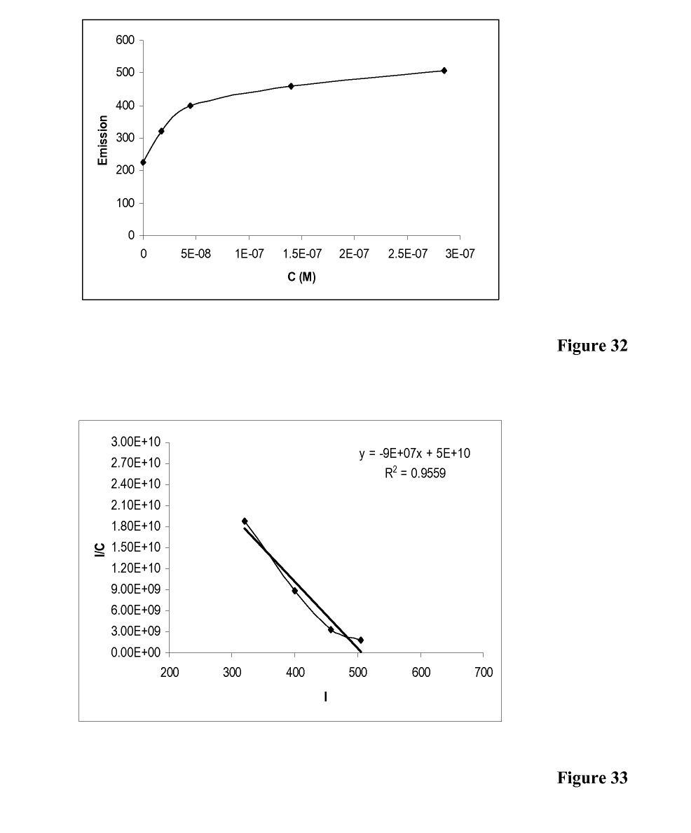

FIG. 32 shows effect of concentration of TNFα on the TNFα antibody functionalized SPN.

FIG. 33 shows Scatchart plot of Tnfa rebinding to Tnfa antibody functionalized SPN

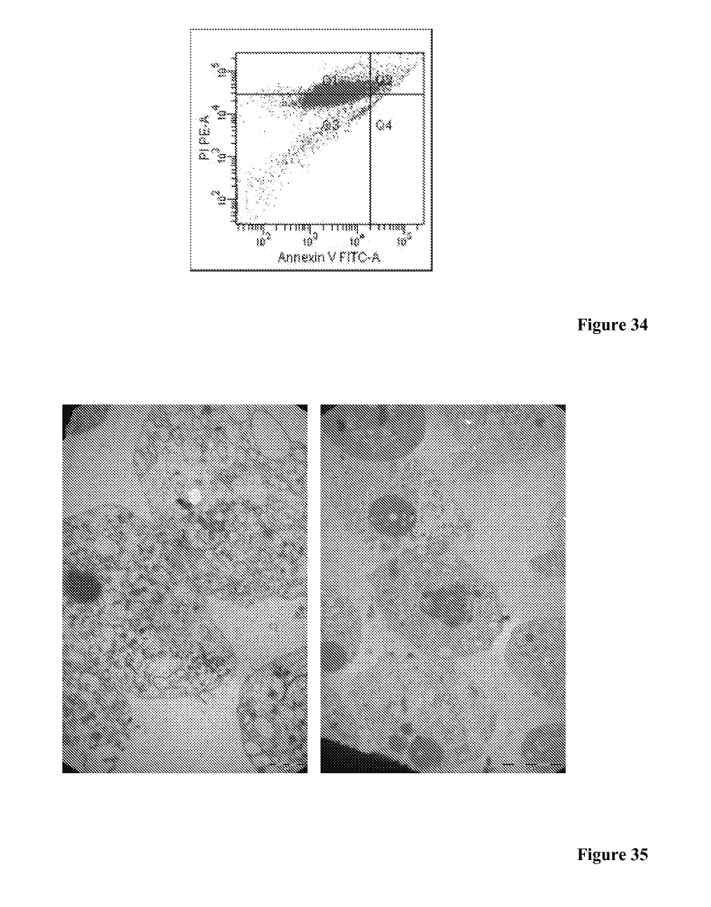

FIG. 34 shows flow-cytometry analyses for (Transferrin-folic acid)-iron oxide particle conjugate and HL60 cells interaction.

FIG. 35 shows TEM images of transferrin and folic acid modified silanized ironoxide nanoparticles and 5RP7 cancer cells interaction.

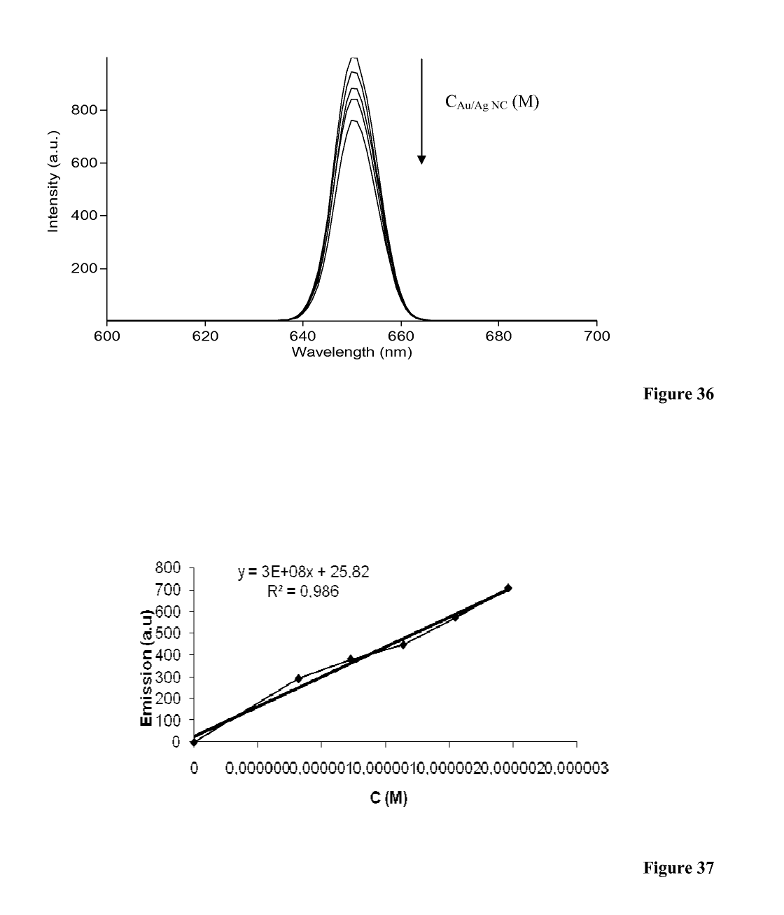

FIG. 36 shows fluorescence spectra of IgG functionalized QDs with the increasing of anti IgG antibody functionalized Au/Ag nanoclusters. The concentration of QDs were 7.7×10⁻⁴ M and that of anti IgG antibody functionalized Au/Ag nanoclusters were 0.0, 5.5×10⁻⁵, 1.64×10⁻⁴, 2.73×10⁻⁴, 5.46×10⁻⁴ M, respectively.

FIG. 37 shows Biotin recognition by biotin imprinted Au/Ag nanoclusters: The effect of concentration of biotin on the biotin imprinted Au/Ag nanoclusters.

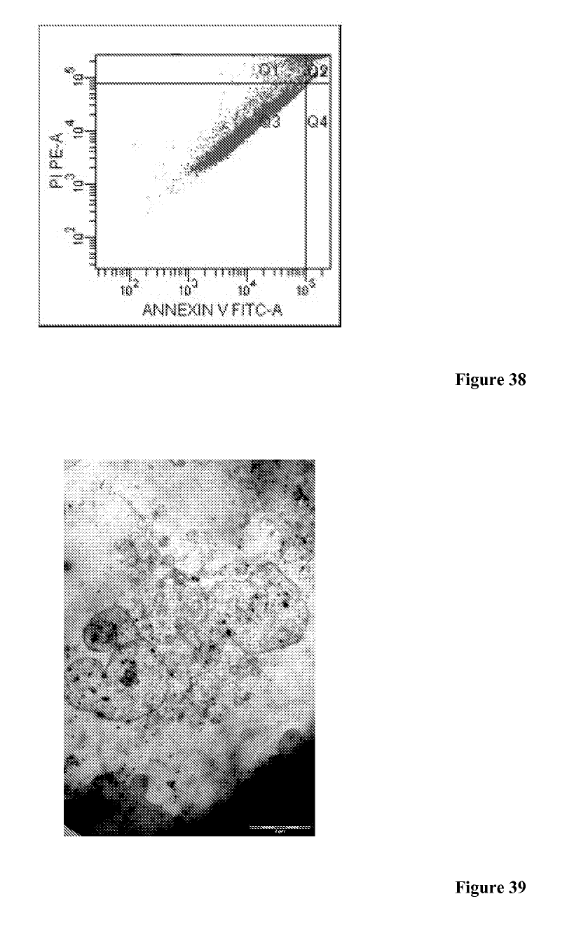

FIG. 38 shows flow cyctometry analysis based on ambush and cell targeting prodrug therapy of transferin decorated and P450 conjugated QD particles and IFA conjugated and anti-transferrin decorated Au—Ag nanoclusters

FIG. 39 shows TEM image of ambush and cell targeting prodrug therapy and HL-60 cancer cells interaction.

FIG. 40 shows SEM images of (A) Lys-MAT doped acrylamide cryogel and (B) Lys-MAT doped acrylamide cryogel with M.Luteus fixation.

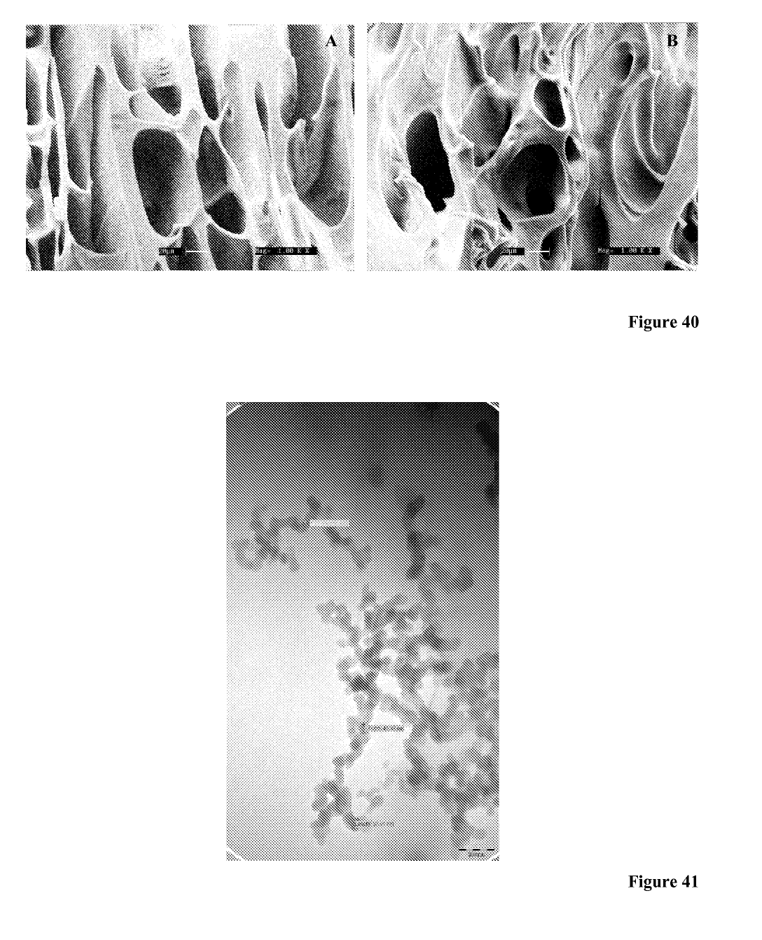

FIG. 41 shows TEM image of photosensitive polymer doped silica nanoparticles

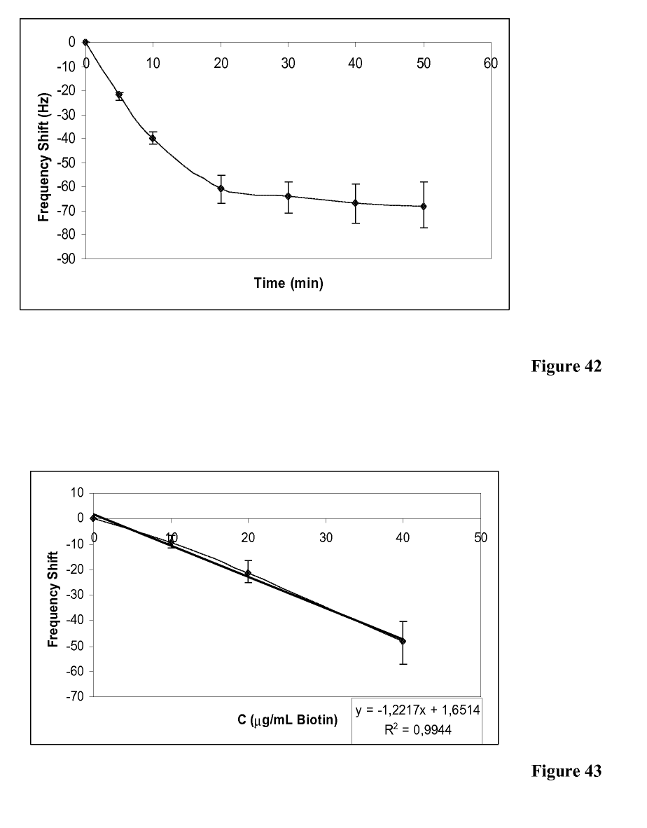

FIG. 42 shows QCM Binding curve for 100 μg mL⁻¹ biotin.

FIG. 43 shows Response of the QCM sensor to biotin concentration

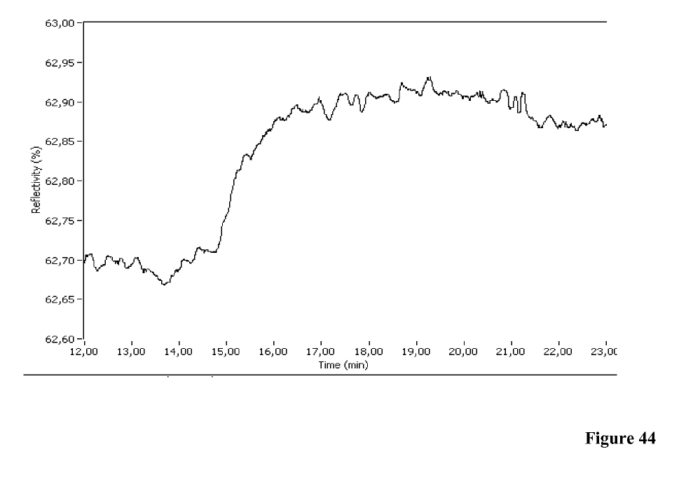

FIG. 44 shows Adsorption isotherms of sialic acid on SPR sensors

Unless defined otherwise, all technical ye scientific terms used herein have the same meaning as commonly understood by one of the familiar skills in the art to which the invention pertains. The following definitions supplement those in the art and are directed to the current applications and are not be imputed to any related or unrelated case, e.g., to any commonly owned patent or application. Although any methods and materials similar or equivalent to those described herein can be used in the practice for testing of the present invention, the preferred materials, platforms and methods are described herein. Accordingly, the terminology used herein is for the purpose of describing particular embodiments only, and is not intended to be confined.

An “amino acid monomer” is monomer linker moiety, i.e. methacryloyl residue having (L)-tyrosine, trytophane or cysteine.

A “a residue” is a methacryloyl residue.

A “photosensitive ruthenium based aminoacid monomer chelate” is a hapten coordination compound that has at least one amino acid monomer as ligand and provides light induced covalent conjugation of biomolecules.

A “photosensitive oligomer (polymer)” is a sequence of ruthenium chelate based amino acid monomers and dissolve in aqua and provides light induced covalent conjugation of biomolecules on to micro and nanoplatforms.

A “biomolecule” is a biological macromolecule and typically a protein (an enzyme, an antibody) that has at least one tyrosine, tyrptophan or cysteine aminoacid.

A “platform” is a micro and nano template for biomolecule as sensor or carrier. Common platforms in the context of the present invention include silica nanoparticles, carbon nanotues, Ag/Au nanoclusters, cadmium sulphide quantum dots, gold surfaces based on quartz crystal or surface plasmon resonanans, super paramagnetic nanoparticules, nanolayers of organoclay and the like.

ANADOLUCA is abbreviation of AmiNoAcid Decorated and Light Underpining Conjugation Approach. It is a novel concept approach which contains the novel syntetic materials and their applications.

Nano-enzyme is an enzyme developed in ANADOLUCA concept. This nano-enzyme is not soluble in solvents where it was activated and reusabled.

A nano-protein is a new generation polymeric material prepared in ANADOLUCA concept. This nano-protein can be use directly as a monomer (enzymes, antibodies or similar proteins) without using any other platforms. Additionally, different kind of drugs and biomolecules can be conjugated to this nano-protein.

[Ru (bipyr)₂L′L″] : Ruthenium based metal-chelate complexes, wherein L′ is an aminoacid monomer (MATyr, MATrp, or MACys), L″ is a chlorine atoms or aminoacid monomers (MATyr, MATrp, or MACys), or benzotriazole having mercaptoundecanoyl.

[Ru (bipyr)₂L′L″]n : Oligomer of Ruthenium based metal-chelate complexes, wherein L′ is an aminoacid monomer (MATyr, MATrp, or MACys), L″ is a chlorine atoms or aminoacid monomers (MATyr, MATrp, or MACys), or benzotriazole having mercaptoundecanoyl.

Conjugation can be defined as the attachment of biomolecules to a micro and nano-surface without affecting conformation and function of protein. In some cases, conjugation may lead to partial or complete loss of protein activity, due to random orientation and structural deformation.

The present invention provides a covalent and photosensitive crosslinking conjugation of the antibodies and other proteins on micro- and nano-structures without affecting conformation and function of proteins through the aminoacid-monomer and/or ruthenium-chelate based aminoacid monomers linkages seems to be ANADOLUCA: the aminoacid-monomer linkages to give covalent bonds with tyrosine, cysteine or a tryptophane of antibodies and other biomolecules and may act as novel nanobioconjugate systems in the field of ligand-receptor interaction investigations for biosensors, synergie between diagnosis and therapie (theranostics) and biocatalysis applications.

Experimental Information

Chemicals were purchased from Aldrich and Sigma and used without further purification. The melting points were determined on Sanyo Gallenkamp. Elemental analyses were performed Vario ELIII. NMR spectra were recorded on a Bruker 500 MHz NMR spectrometer Ultra-shield FT-NMR spectrometer at room temperature. All MALDI-TOF/MS mass spectra were acquired on a Voyager Biospectrometry STR Workstation/the system utilizing a pulsed nitrogen laser, emitting 337 nm. The acceleration voltage was set to 20 kV and the delay time was 100 ns. Mass analysis was carried out in positive reflector mode and delayed extraction mode applied. a-cyano-4-hydroxycinnamic acid (CHCA) matrix solution was used. 10 mg CHCA solved in 1:1, 1 mL of 0.3% TFA solution and acetonitrile. 2 μL sample solution was mixed with 23 μl of a 10 mgmL⁻¹ solution of CHCA in acetonitrile/0.3% TFA. This preparation (1 μL) was placed onto a MALDI-TOF/MS sample plate and allowed to dry. Photo-luminescence spectra were obtained using a Carry Eclipse Varian Model Fluorescence Spectrometer. TEM samples were prepared and TEM (BioTwin G² Sprint 120 kV) was used for imaging of samples. Imaging works were obtained using Leica Fluorescence Microscope. Flow-Cytometry experiments were obtained FACS Aria (BD Bioscience) flow cytometer using the Diva software.

### Chemical Syntheses of Ruthenium Based Photosensitive Aminoacid Monomers and Oligomers

According to the present invention, a monomer having the function of a photosensitive bio-conjugator is provided, the monomer is oligo (or poly)-merized to prepare photosensitive oligo(poly)mer and/or the photosensive polymer is used to conjugate the proteins on to micro and nano-platforms. The monomer according to the present invention is a novel methacryloyl and ruthenium based monomer having at least one aminoacid monomer ligand which conjugates proteins to a platforms. The ruthenium-chelate based monomers include methacryloyl incorporating tyrosine, or trytophane as ligand and/or chlor or MUABt and other ligands, which can be presented by the following Formula 1-9.

Chemical Synthesis of the Aminoacid-Monomer

3-(4-hydroxyphenyl)-2-[(2-methylacryloyl)amino]propanoic acid (methacryloyl tyrosine, (MATyr)), N-[1-(1H-Indol-3-ylmethyl)-2-oxo-propyl]-2-methylacrylamide (methacryloyl tryptophan, (MATrp)), 2-[(2-methylacryloyl)amino]-3-sulfanylpropanoic acid (methacryloyl cysteine, (MACys)), and 5-carbamimidamido-2-[(2-methylacryloyl)amino]pentanoic acid (methacryloylarginine, (MAArg)) were prepared according to following published method (Deniz Hür, Sultan F. Ekti and Ridvan Say, Letters in Organic Chemistry, 2007, 4, 585-587) by us.

An appropriate amino acid (1 eq.) was dissolved in 1 M aqueous solution of NaOH (1 eq.) in a round bottom-flask. A solution of methacryloyl benzoriazole (MA-Bt) (1 eq) in 25 mL of 1,4-dioxane was slowly added to the amino acid solution. Reaction mixture was allowed to stir for 10-20 min. at room temperature. Completion of reaction is monitored by TLC, after the reaction finished, 1,4-dioxane was evaporated under vacuum. The residue was diluted with water and extracted with ethyl acetate (3×50 mL) to remove 1H-benzotriazolee. Collected water phases were neutralized to pH=6-7 using 10% water solution of HCl (pH should keep around 6-7 to prevent possible polymerization of methacryloyl group in acidic medium). Water was removed via rotary-evaporator to give MATyr in 90% , MA-Trp in 85%, MA-Arg in 84 and MACys in 83% yield.

Data for MA-Trp

¹H NMR (500 MHz, DMSO-d6), δ: 10.7 (s, 1H), 7.45 (d, 1H, J=7.89 Hz), 7.22-7.38 (m, 2H), 6.96-7.1 (m, 2H), 6.87 (t, 1H, J=7.40, 14.86 Hz), 5.48 (s, 1H), 5.21 (s, 1H), 4.05 (dd, 1H, J=4.80, 9.70 Hz), 3.28 (dd, 1H, J=4.60, 14.16 Hz), 3.09 (dd, 1H, J=4.60, 14.16 Hz), 1.78 (s, 3H) ppm.

¹³C (125 MHz, DMSO-d6), δ: 19.0, 27.5, 55.5, 111.5, 112.0, 118.5, 119.0, 119.5, 121.0, 124.0, 129.0, 136.0, 141.0, 166.0, 173.5 ppm.

Data for MATyr

¹H (500 MHz, DMSO-d6), δ: 7.35 (d, J=8.05 Hz, 2H), 7.05 (d, J=8.40 Hz, 2H), 6.29 (s, 1H), 5.90 (s, 1H), 3.15 (dd, J=4.49, 14.25 Hz, 1H), 3.02 (dd, J=4.02, 14.12 Hz, 1H), 2.90 (dd, J=7.75, 14.20 Hz, 1H), 2.00 (s, 3H) ppm.

¹³C (125 MHz, DMSO-d6), δ: 170.9, 165.8, 149.6, 135.9, 130.9, 128.0, 121.8, 115.6, 55.9, 37.0, 18.5 ppm.

Data for MACys

¹H (500 MHz, DMSO-d6 and D₂O): Because of the solubility problem of MAC, NMR spectra fort his compound was recorded both in DMSO-d6 and D₂O. δ: In DMSO-d6 two single peaks between 7.67-7.36 ppm were recorded and since these peaks were disappeared in D₂O, it is assumed that these are —SH and —NH peaks. 5.67 (s, 1H) and 5.31 (s, 1H) peaks were assigned to the etilen group of methacryloyl group. 4.16 (m, 1H) was assigned to the CH proton bonded to the NH group of amino acid. (—CH₂—CH—NH—). 3.10-3.02 (m, 2H) peak was assigned to the —CH₂— protons for amino acid. 1.88 (s, 3H) was assigned to the —CH₃ group on methacryloyl moiety.

¹³C (125 MHz, D₂O), δ: 177.5, 172.9, 141.8, 122.6, 55.1, 30.1, 17.49 ppm

Data for MA-Arg

¹H-NMR (500 MHz, D₂O), δ: 5.60 (s, 1H, CH₂═C(CH₃)—), 5.35 (s, 1H, CH₂═(CH₃)—), 3.95 (dd, 1H, J=4.54 Hz, 6.71 Hz, HOCH₂—CHNH—), 2.92-2.85 (m, 2H, —CH₂—CH₂—NH—),

¹³C (125 MHz, D₂O), δ: 17.9, 35.0, 45.1, 50.0, 50.2, 122.0, 163.1, 173.0, 175.0, 181.0 ppm.

Synthesis of MUA-Bt as Ligand

11-(1H-benzotriazole-1-yl)-11-oxoundecane-1-thiol(MUA-Bt) was prepared according to following literature method (A. R. Katritzky, Y. Zhang, S. K. Singh, Synthesis 18 (2003) 2795-2798.)

Thionylchloride (SOCl₂) (1 eq) was added to solution of 1H-Benzotriazolee (Bt-H) (3 eq) in CH₂Cl₂ (100 mL) under nitrogen atmosphere at room temperature. After stirring 30 min, 11-mercaptoundecanoic acid (MUA) was poured into the reaction mixture. Upon the addition white precipitate was formed. The reaction mixture was stirred 3 h. After this perion white solid was filtered and washed with CH₂Cl₂. The collected filtrate was extracted with 2 M NaOH (3×50 mL) to remove excess of Bt-H. The organic layer was dried with MgSO₄, the solvent was removed under vacuum. The crude product was purified using ethyl acetate-hexane mixture over silicagel column to get MUA-Bt as white microcrystals in %87 yield.

¹H NMR (500 MHz, CDCl₃-d), δ: 8.32 (d, J=8.27 Hz, 1H), 8.15 (d, J=8.30 Hz, 1H), 7.68 (t, J=7.15 Hz, 1H), 7.53 (t, J=7.17 Hz, 1H), 3.44 (t, J=7.50 Hz), 2.89 (t, J=7.50 Hz, 1H), 2.70 (t, J=7.50 Hz, 1H), 1.89-1.97 (m, 2H), 1.66-1.79 (m, 2H), 1.65-1.160 (m, 1H), 1.54-1.46 (m, 2H), 1.44-1.37 (m, 4H), 1.35-1.28 (m, 6H) ppm.

13C NMR (125 MHz, CDCl₃-d), δ: 24.5, 28.5, 28.8, 29.1, 29.2, 29.4, 29.4, 35.5, 38.9, 39.2, 114.5, 120.1, 126.1, 130.3, 131.14, 146.2, 172.7 ppm.

Chemical Synthesis of the Photosensitive Ruthenium Based Aminoacid-Monomer

Dichlorobis(2-2’-bipyridyl)ruthenium(H) : 1 eq RuCl₂(MeSO)₄ and 2 eq 2-2′-bipyridyl were refluxed in chloroform (30 mL) for ca. 1 h. The solvent was removed and the residue dissolved in acetone. Addition of ether precipitated the orange complex. This was filtered off, washed with ether and dried under vacuum [Rempel et al.]. M. p.: 205-210° C.

Anal. for C₂₀H₁₅Cl₂N₄Ru: found: C 48.20%, H 3.01%, N 12.89%, calcd.: C 49.70%, H 3.13%, N 11.59%

¹H NMR (500 MHz, CDCl₃), ppm: 10.25 (d, 2H, J=4.35 Hz), 9.93 (d, 2H, J=4.29 Hz), 9.73 (d, 2H, J=5.86 Hz), 9.65 (d, 2H, J=4.52 Hz),

MALDI-TOF-MS: The ion peaks at 79, 128 and 156 m/e relating to bipyridyl. m/e 101, 413 shows Ru and Ru(bpy)₂ respectively. m/e 293, 327, 448 and 484 relating to RubpyCl, RubpyCl₂, Ru(bpy)₂Cl and Ru(bpy)₂Cl₂ respectively. The MS-spectrum data confirm that Ru(bpy)₂Cl₂ structure was produced exactly.

Chlorobis(2-2’-bipyridyl) MATrp-ruthenium(H) (1): 1 eq RuCl₂(bipyr)₂ was dissolved in water. The solution cooled to 0° C. and added NEt₃. the aqueous solution of 1 eq MATrp was added dropwise that solution and stirred at room temperature for 30 min. The mixture was heated to 80° C. for refluxing ca. 24 h. The brown complex (FIG. 1) which separated was filtered off, washed with ether and dried under vacuum. M. p.: 170-172° C.

Anal. for C₃₅H₃₁ClN₆O₃Ru: found: C 57.77%, H 4.22%, N 13.14%, calc.: C 58.37%, H 4.34%, N 11.67%

MALDI-TOF-MS: The ion peaks at 79, 128 and 155 m/e relating to bipyridyl. m/e 136, 293, 413 and 448 relating to RuCl, RubpyCl, Ru(bpy)₂, and Ru(bpy)₂Cl respectively. m/e 272, 408, 529 and 564 relating to MATrp, RuMATrpCl, RubpyMATrp, RubpyMATrpCl. This data confirm that MATrpRu(bpy)₂Cl structure was produced exactly.

Bis(2-2′-bipyridyl)bis(MATrp)-ruthenium(II) (2): RuCl₂(bipyr)₂ (1 eq) was dissolved in water. The solution cooled to 0° C. and added NEt₃. The aqueous solution of MATrp (2 eq.) was added dropwise that solution and stirred at room temperature for 30 min. The mixture was heated to 8020 C. for reflaxing ca. 24 h. The brown complex (FIG. 2) which separated was filtered off, washed with ether and dried under vacuum. M. p.: >200° C.

Anal. for C₅₀H₄₆N₈O₆Ru: found C 61.97%, H 4.3%, N 10.8. calcd C 62.82%, H 4.85%, N 11.72%

¹H NMR (500 MHz, CDCl₃), ppm: 10.74 (d, 2H, J=14.9 Hz), 9.76 (s, 1H), 7.99 (d, 1H, J=7.68 Hz), 7.91 (d, 1H, J=7.57 Hz), 7.54 (d, 2H, J=7.94 Hz), 7.46 (d, 1H, J=8.2 Hz), 7.31 (t, 4H, J=6.76 Hz), 7.16 (s, 1H), 7.1 (d, 2H, J=11.7 Hz), 7.07-7.00 (q, 5H, J=8.0 Hz), 5.6(d, 2H, J=6.6 Hz), 5.3 (s, 2H), 4.57(m, 1H, 4.39(m, 2H), 1.24 (s, 3H).

MALDI-TOF-MS: The ion peaks at 79 and 155 m/e relating to bipyridyl. m/e 272, 373, 413, 529 and 685 relating to MATrp, Ru-MATrp, Ru(bpy)₂, Ru(bpy)-MATrp and Ru(bpy)₂ MATrp respectively.

Bis(2-2′-bipyridyl)-MATrp-MUABt ruthenium(H) (3—FIG. 3): 1 eq 1 was dissolved in MetOH. 1 eq MUABt was dissolved in DMSO and added to the first solution by dropwise at room temperature. Mixture was stirred at 110° C. for 6 h. Solvents were removed under reduced pressure and residue taken in CH₂Cl₂. Organic layer washed with H₂O (3×10 mL), dried over MgSO₄, washed with ether and dried under vacuum. M. p.: >220° C.

Anal. for C₅₂H₅₅N₉O₄RuS: found: C 61.53%, H 4.92%, N 12.99%, calcd.: C 62.26%, H 5.53%, N 12.57%.

¹H NMR (500 MHz, CDCl₃), ppm: 8.32 (d, 1H, J=9.72 Hz), 8.14 (d, 1H, J=8.26 Hz), 7.89 (s, 2H), 7.67 (t, 1H, J=7.66 Hz), 7.52 (t, 1H, J=7.65 Hz), 7.41 (d, 2H, J=5.86 Hz), 3.68 (s, 10H), 3.44 (t, 2H, J=7.48 Hz), 2.88 (t, 6H, J=7.33 Hz), 2.33 (m, 16H), 1.92 (p, 3H), 1.74 (t, 8H, J=7.35 Hz).

MALDI-TOF-MS: The ion peaks at 79, 128 and 155 m/e relating to bipyridyl. m/e 101, 413 shows Ru and Ru(bpy)₂ respectively. m/e 272 and 318 peaks show MATrp and MUABt monomers. m/e 419, 529 and 1003.1 peaks show Ru -MUABt, Ru(bpy)-MATrp and Ru(bpy)₂MATrp-MUABt.

Chlorobis(2-2′-bipyridyl) MATyr-ruthenium(II) (4—FIG. 4): 1 eq RuCl₂(bipyr)₂ was dissolved in methanol. The solution cooled to 0° C. and added NEt₃. 1.2 eq MATyr in methanol solution was added dropwise that solution and mixture refluxed at 55° C. for 48 h. At the end of the reaction time, solvent was removed under reduced pressure and residue was dissolved in dichloromethane. Solution was washed with 3× H₂O and dried with MgSO₄. After that solvent evaporated the product washed with ether and dried under vacuum. M. p.:125-128° C.

Anal. for C₃₄H₃₃ClN₅O₄Ru: found: C 56.75%, H 5.01%, N 10.23%, calcd.: C 57.34%, H 4.67%, N 9.83%.

¹H NMR (500 MHz, CDCl₃), ppm: 9.7 (d, 4H, ³J=5.08 Hz), 8.56 (d, 4H, ³J=7.92 Hz), 7.92 (t, 4H, ³J=7.82 Hz), 7.3 (t, 1H, ³J=7.4 Hz), 7.23 (t, 2H, ³J=7.47 Hz), 7.18 (d, 2H, ³J=7.46 Hz), 7.12 (t, 4H, ³J=6.30 Hz), 5.3 (d, 1H, ²J=1.52 Hz), 5.3 (d, 1H, ²J=1.54 Hz), 1.8 (s, 3H)

MALDI-TOF-MS: The ion peaks at 79 m/e relating to bipyridyl. m/e 101, 413 and 448 data show Ru, Ru(bpy)₂ and Ru(bpy)₂Cl respectively. m/e 250 and 351 peaks show MATyr monomer and Ru-MATyr respectively.

Bis(2-2’-bipyridyl)bis(MATyr)-ruthenium(II) (5): RuCl₂(bipyr)₂ (0.1 gr, 1 eq.) was dissolved in water. The solution cooled to 0° C. and added NEt₃. The aqueous solution of MATyr (0.1 gr, 2 eq.) was added dropwise that solution and stied at room temperature for 30 min. The mixture was heated to 80° C. for reflaxing ca. 24 h. The brown complex (FIG. 5) which separated was filtered off, washed with ether and dried under vacuum. M. p.: >200° C.

Anal. for C₄₆H₄₄N₆O₈Ru: found: C 61.54%, H 4.63%, N 10.56%, calcd.: C 60.72%, H 4.87%, N 9.24%

¹H NMR (500 MHz, CD₃OD), ppm: 9.78 (1H, s), 7.8 (1H, d, J=8.15 Hz),7.73 (t, 1H, J=4.1 Hz), 7.64 (t, 2H, J=4.11 Hz), 7.47-7.41 (p, 2H), 7.3-7.11 (m, 11H), 7.05 (t, 3H, J=8.18 Hz), 6.95 (d, 2H, J=8.18 Hz), 6.78 (s, 9H), 6.69 (d, 1H, J=12.75 Hz), 6.34 (s, 1H), 5.84 (s, 1H), 4.5 (s, 1H), 4.25 (s, 2H), 2.06 (s, 3H), 1.91 (s, 3H).

MALDI-TOF-MS: The ion peaks at 79, 128 and 155 m/e relating to bipyridyl. m/e 101, 413 peaks show Ru and Ru(bpy)₂ respectively. m/e 250, 599 and 755 data show MATyr monomer, Ru-(MATyr)₂ and -Ru(bpy)-MATyr complex.

Bis (2-2’-bipyridyl) MATyr-MATrp-ruthenium(II) (6): 1 eq 4 was dissolved in methanol. 1.2 eq MATrp solution in methanol was added by dropwise at room temperature. Mixture was refluxed at 80° C. for 24 h. At the end of the reaction time solvent was removed under reduced pressure and residue was dissolved in dichloromethane. Solution was washed with 3× H₂O and dried with MgSO₄. After that solvent evaporated the product (FIG. 6) washed with ether and dried under vacuum. M. p.:110-112° C.

Anal. for C₄₉H₄₉N₇O₇Ru: found: C 61.56%, H 4.9%, N 11.67%, calcd.: C 62.01%, H 5.2%, N 10.33%.

¹H NMR (500 MHz, CDCl₃), ppm: 9.4 (d, 2H, ³J=5.2 Hz), 8.52 (d, 2H, ³J=8.08 Hz), 8.1 (s, 4H), 7.98 (t, 4H, ³J=7.74 Hz), 7.62-7.01 (m, 11H), 5.71 (d, 1H, ²J=1.57 Hz), 5.43 (d, 1H, ²J=1.52 Hz), 4.7 (t, 1H, ³J=5.7 Hz), 1.9 (s, 6H)

MALDI-TOF-MS: The ion peaks at 250 and 272 relating to MAT and MATrp monomer. m/e 257 and 413 data show Ru-bpy and Ru(bpy)₂ respectively. m/e 351, 529 and 622 peaks show Ru-MAT, Ru-bpy-MATrp and Ru-MATyr-MATrp respectively.

Bis(2-2′-bipyridyl)-MATyr-MUABt ruthenium(II) (7): 1 eq 4 was dissolved in DMSO. 1.2 eq MUABt was dissolved in DMSO and added to the first solution by dropwise at room temperature. Mixture was stirred at 110° C. for 24 h. 20 mL H₂O was added at reaction mixture and solution was extracted with CH₂Cl₂ (3×10 mL). Solvent was removed under reduced pressure and brown complex (FIG. 7) dried under vacuum. M. p.: 170° C.

Anal. for C₅₀H₅₅N₈O₅RuS: found: C 60.02%, H 6.21%, N 11.89%, calcd.: C 61.21%, H 5.65%, N 11.42%.

¹H NMR (500 MHz, CDCl₃), ppm: 10.3 (d, 2H, ³J=5.1 Hz), 9.39 (d, 1H, ³J=8.08 Hz), 8.9 (d, 1H, ³J=7.89 Hz), 8.4 (d, 1H, ³J=8.15 Hz), 8.11 (t, 4H, ³J=7.7 Hz), 7.92 (t, 1H, ³J=7.78 Hz), 7.40 (t, 4H, ³J=6.27 Hz), 7.22 (t, 2H, ³J=7.36 Hz), 7.18 (d, 2H, ³J=7.34 Hz), 6.18 (t, 2H, ³J=6.56 Hz), 5.3 (d, 1H, ²J=1.5 Hz), 5.28 (d, 1H, ²J=1.5 Hz), 3.2 (t, 2H, ²J=17. Hz), 2.5 (q, 2H, ²J=13 Hz), 1.87 (s, 3H).

MALDI-TOF-MS: The ion peaks at 79 and 155 m/e relating to bipyridyl. m/e 101, 413 data show Ru and Ru(bpy)₂ respectively and m/e 250 and 575 data show MATyr monomer and MUABt-Ru(bpy) complex.

Chlorobis (2-2’-bipyridyl) MACys-ruthenium(II) (8): 1 eq RuCl₂(bipyr)₂ was dissolved in ethanol. The solution cooled to 0° C. and added NEt₃. 1.2 eq MACys in ethanol solution was added dropwise that solution and mixture refluxed at 55° C. for 48 h. At the end of the reaction mixture was filtered and organic solvent removed under reduced pressure. Brown oil residue was dissolved in dichloromethane. Solution was washed with 3× H₂O and dried with MgSO₄. After that solvent evaporated the product (FIG. 8) washed with ether and dried under vacuum. M. p.:105-108° C.

Anal. for C₂₉H₃₃ClN₅O₃SRu: found: C 53.05%, H 4.85,%, N 10.87%, calcd.: C 52.13% H 4.98%, N 10.48%.

¹H NMR (500 MHz, CDCl₃), ppm: 9.55 (d, 4H, ³J=5.08 Hz), 8.45 (d, 4H, ³J=8.03 Hz), 7.97 (t, 4H, ³J=7.52 Hz), 7.50 (t, 2H,³J=5.13 Hz), 7.05 (t, 4H, ³J=6.78 Hz), 5.72 (d, 1H, ²J=1.52 Hz), 5.61 (t, 1H, ³J=4.28 Hz), 5.3 (d, 1H, ²J=1.53 Hz), 2.9 (t, 2H, ²J=14.64 Hz), 1.96 (s, 3H)

MALDI-TOF-MS: m/e 413 and 448.6 data show Ru(bpy)₂ and Ru(bpy)₂Cl respectively. m/e 276, 301, 467 and 623 relating to Ru-MACys, Ru-MACysCl, Ru(bpy)-MACys-Cl and Ru(bpy)₂MACys-Cl respectively. This data confirm that Ru(bpy)₂MACysCl structure was produced exactly.

Bis (2-2’-bipyridyl)bis(MACys)-ruthenium(II) (9): 1 eq 8 was dissolved in ethanol. The solution cooled to 0° C. and added NEt₃. 1.2 eq MACys solution in ethanol was added by dropwise at room temperature. Mixture was refluxed at 75° C. for 48 h. At the end of the reaction mixture was filtered, solvent was removed under reduced pressure and residue was dissolved in dichloromethane. Solution was washed with 3× H₂O and dried with MgSO₄. After that solvent evaporated the product (FIG. 9) washed with ether and dried under vacuum. M. p.:92-94° C.

Anal. for C₃₇H₄₇N₆O₆S₂Ru: found: C 53.97%, H 5.83,%, N 9.93%, calcd.: C 53.09%, 5.66%, N 10.04%.

¹H NMR (500 MHz, CDCl₃), ppm: 9.65 (d, 4H, ³J=5.04 Hz), 8.40 (d, 4H, ³J=7.96 Hz), 7.92 (t, 4H, ³J=7.7 Hz), 7.52 (t, 2H, ³J=5.02 Hz), 7.03 (t, 4H, ³J=6.80 Hz), 5.72 (d, 2H, ²J=1.55 Hz), 5.61 (t, 2H,³J=4.45 Hz), 5.3 (d, 2H, ²J=1.54 Hz), 2.9 (4H, t, ²J=13.8 Hz), 1.89 (s, 6H)

MALDI-TOF-MS: m/e 257 and 413 data show Ru(bpy) and Ru(bpy)₂ respectively. m/e 432 and 763 relating to Ru(bpy) MACys and (MACys)₂Ru(bpy)₂ respectively. This spectrum confirms that Ru(bpy)₂(MACys)₂ structure was produced exactly.

Preparation of the Photosensitive Oligo (Poly)mer

The photosensitive oligo(poly) mer according to the present invention is the oligomer including the repeating unit produced from photosensitive ruthenium based aminoacid-monomers and can be represented by the following Reaction 1. The phosensitive oligomer of Reaction 1 can be prepared by a conventional polymerization reaction. For example, the photosensitive oligomer of Reaction 1 can be prepared according to the following polymerization Reaction 1. As shown in Reaction 1, the monomers for preparing the photosensitive oligomer, such as Bis(2-2’-bipyridyl)-MATrp-MATyr ruthenium(II) monomer of Formula (FIG. 6) and other kinds of ruthenium based aminoacid monomers, are mixed by necessary amounts in organic solvent, and subjected to a polymerization reaction to obtain a reaction product. Exemplary organic solvent for the polymerization reaction includes acetone, dimethyl sulfoxide, dimethyl formamide, dioxane, acetonitrile, methanol, and so on. The obtained reaction product can be crystalized in an organic solvent such as tetrahydrofurane (THF) to produce the photosensitive oligomer (water dissolve) of the present invention. The polymerization reaction is preferably carried out in the presence of an initiator. Exemplary initiator includes 2-2′-azobisisobutyronitrile (AIBN), benzoyl peroxide (BPO), APS, and so on. These polymers contain about 15-24 repeating units according to MALDI-TOF-MS analyses.

For example, oligomer of Bis(2-2′-bipyridyl)-MATrp-MATyr ruthenium(II) has 19 monomer units (FIG. 10).

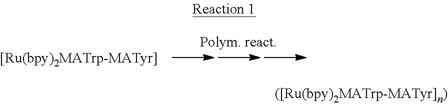

Instrument setting: 2 μL ([Ru(bpy)₂MATrp-MATyr]ₙ) solution was mixed with 23 μL of a 10 mgmL⁻¹ solution of CHCA in acetonitrile/0.3% TFA. The acceleration voltage was set to 20 kV, the delay time was 400 ns, grid voltage % 70, laser intensity 2092 and at reflector mode.

Comments: The ion peaks at 250, 351, 506, 529, 662 and 685 m/e relating to MATyr monomer, Ru-MATyr, Ru-bpy-MATyr, Ru-bpy-MATrp, Ru(bpy)₂MATyr, Ru(bpy)₂ MATrp respectively. m/e 155, 413 and 448 shows bpy, Ru(bpy)₂ and Ru(bpy)₂Cl respectively. m/e 172, 190, 212 and 379 relating to CHCA matrix.

### EXAMPLES

Hereinafter, the preferable examples are provided for better understanding of the present invention and ANADOLUCA concept and its scope of the appended claims. Accordingly, the following examples are offered to illustrate, but not to limit, the claimed invention. It will be understood that methods using bioconjugation strategies based on photosensitive aminoacid monomer linkage on to micro and nano-platforms according to this invention may also be useful in therapie and diagnosis and imaging as nanocargoes in the pharmaceutical, medicinal and diagnostic industry, and in enzyme based biotechnology in energie industry, food and agriculture and click chemistry.

### Example 1

Preparation of Protein Nanocargoes and Its Conjugates

The following sets forth a series of experiments that demonstrate preparation of exemplary nanocargo proteins and its conjugates through ANADOLUCA concept.

The most commonly used carriers are highly immunogenic. The criteria for a succesful carrier molecule are the potential for immunogenicity and lack of toxicity in vivo. This invention describes preparation and use of protein nanocarrier platforms (nanocargoes) that are designed to have low immunogenicity on their own to minimize the potential for antibody against them and to have cell receptors for targeting, drugs for therapie and imaging molecule and/or photosensitive hapten conjugates.

The resultant conjugates are important in bioconjugation research, nanotheranostic technology, drug targeting research, antibody purification and in immune research and creation of vaccines.

Preparation of BSA Nanocargo and Its Conjugates

The one of most common carrier proteins in use today is BSA. In other hand, small molecules like tyrosine, tyrotophane, cysteine or photosensitive ruthenium based aminoacid monomer chelates in the invention, as they are called, haptens, can be made immunogenic by coupling them to nanocargoes and at the same time, photosensitive cross-linking and bioconjugation of other proteins like transferrin, HRP.

Polymeric particles can be constructed from a number of different monomers or copolymer combinations. In these examples of the invention, by mixing into the polymerization reaction combinations of aminoacid monomer like MATyr, MATrp, MACys or photosensitive ruthenium based aminoacid monomer having chelate and carrier protein like BSA and IgG monomers, one can create BSA groups as the monomer on the nanoparticle surface for subsequent coupling to haptens or other proteins like receptors. These carrier protein having polymeric nanoparticles are protein nanospheres, probably from the definition of an “emulsion of protein-rubber”. These polymeric protein nanoparticles through photosensitive emulsion polymerization consist of polyaminoacid or ruthenium based polyaminoacid copolymers of protein like/MATyr/protein, MATrp/protein and MATyr-Ru (bpyr)₂-MATyr/protein and its copolymer forms having ethylenglycol dimethacrylate (EDMA).

The example describes the synthesis of the BSA nanocargo having BSA and aminoacid monomers like MATyr (or MACys and/or MATrp) by photosensitive microemulsion polymerization technique. 0.5 g polyvinyl alcohol (PVA) was dispersed in 45 mL deionized water to form microemulsion system. In the synthesis, 25 mL of this dispersed solution of PVA was used. On the other hand, 0.5 g MATyr was dissolved in 2 mL dimethylsulfoxide to prepare MATyr solution. 4 mg BSA was dissolved in 2 mL deionized water and then added into MATyr solution. After that, 40-250 μL of 1.0 mg mL⁻¹ photosensitive ruthenium based oligomer or bis(2-2′-bipyridyl)ruthenium (II) aqueous solution was added into this mixture. Included MATyr, photosensitive solution and BSA mixture was added into PVA dispersion media and mixed for 20 minutes. Then, 0.3 mL crosslinker agent, EDMA was added in solution and mixed for 5 min. Lastly, 0.02 g ammonium peroxodisulphate (APS) was dissolved in 45 mL deionized water to prepare initiator solution and it was added into the microemulsion media to start the polymerization. Prepared solution was mixtured on a magnetic mixture for 48 h at 150 rpm, room temperature under nitrogen atmosphere at daylight. At the end of 48 h these prepared BSA nanoparticles was centrifuged at 6000 rpm for 20 min then precipitated BSA nanoparticles taken away from the solution media and washed with deionized water for two times. BSA nanoparticles was stored at 0° C. until used.

The nano BSA particles are spherical and between 60-150 nm in diameter. And both natural BSA and polymeric nano BSA have autofluorescens in the near ultraviolet.

HRP-conjugated nano BSA cargoes for imaging and sensing

BSA is a single polypeptide chain consisting of about 583 amino acid residues and no carbohydrates. In this example, we investigated the interaction between HRP and the serum albumin protein nanocargo as explained in Example [80]. HRP bound to nano BSA particles in presence of 1-Ethyl-3-[3-dimethylaminopropyl]carbodiimide hydrochloride/N-hydroxysulfosuccinimide (EDC/NHS-to prepare stable NHS ester derivates.

Carbodiimides are used to mediate the formation of amide linkages between carboxylates and amines (Hoare, D.G., Koshland, D.E., (1966) J. Am. Chem. Soc., 88, 2007). The single-step method using EDC alone is appropriate for use in coupling molecules having one or more amines present without any carboxylates. If the molecule being coupled has both amines and carboxylates, such as proteins, then it best the two-step method. Carboylate or carboxylate having particles can be coupled to amine-containing molecules using a number of reaction strategies. The most frequently used method involves an aqueous two-step coupling process using EDC and NHS or sulfo-NHS to form amide bond with a protein or other molecules (Greg T. Hermansson, (2008) Bioconjugate Techniques (Second Edition) Elsevier).

While the carboxyl-containing BSA could be made to react directly with the amine-containing molecule by the addition of EDC, the reaction was much more efficient with Sulfo-NHS present because a stable intermediate was created. Then, HRP was conjugated to the nanoBSA-EDC/NHS mixture through amide linkage of amines of HRP aminoacids. On the other hand, amines of nanoBSA aminoacids can be activated by carboxylate of photosensitive monomer haptens like MATyr, MATrp, MACys, MATyr-Ru(bpyr)₂-MATyr, MATrp-Ru(bpyr)₂-MATrp, MACys-Ru(bpyr)₂-MACys through amide bond formation in presence of EDC which reacts carboxylic groups of these aminoacid monomer haptens and the photosensitive aminoacid monomer hapten labeled BSA nanocargo to results in bio conjugation and cross-linking of HRP,

The enzyme horseradish peroxidase (HRP) reacts with its substrate, hydrogen peroxide, in the presence of an electron donor (i.e. diamino-benzidine) to yield a brownish reaction product. This reaction product can be visualized by light microscopy. 3,3′-diaminobenzidine (DAB) was used as the substrate in HRP catalysis reaction. Firstly, BSA nanocargo was modified with aminoacid monomer hapten in presence of EDC (described as [above) and then HRP solution was added with the same amount of aminoacid monomer hapten labeled BSA nanocargo in presence of APS and day light. Reaction was continued on magnetic mixture for 12 h. This provides a strategy to investigate the interaction between HRP conjugated and cross-linking BSA nanocargo and H₂O₂/DAB through the evaluation of specific parameters like spectrometric analysis and visualized that describe the binding process.

In this example, BSA which has been used like a nanocargo, was labeled with photosensitive aminoacid monomer hapten in presence EDC and then HRP was added to give covalent bonds with tyrosine, cysteine or a tryptophane of transferrin through photopolymerization in presence of APS for targeting and labeling. These provides a strategy to investigate the interaction between BSA and HRP through the evaluation of specific parameters like UV spectroscopy analysis.

DAB working solution was prepared by dissolving 0.002 g DAB in distilled water and diluted to 2.0 mL (5 mM). The solution of HRP (from Sigma) was prepared by dissolving 2 mg HRP in 5 ml distilled water, and then was stored at 4° C. Deionized water was added to 10 mg BSA nanocargoes for 200 ppm preparation of disperse solution. 250 μl EDC 0.2 M was added to BSA nanocargo dispersed solution. And then, 100 μl of 0.5 mg mL⁻¹ MATrp-Ru(bipyr)₂-MATrp hapten aqueous solution was added into BSA nanocargo for labeling of BSA nanocargo in the presence of APS and day light and 200 ppm HRP was prepared with deionized water and 500 μl HRP solution was added into aminoacid monomer hapten conjugated BSA nanocargo dispersed solution. After that mixture was mixed for 12 h at the same condition of example describe as above. After 12 h, HRP conjugated BSA mixture was centrifuged so non binding HRP was removed and then, sediments were taken and washed with dionized water. Lastly, washing water was removed by centrifuged.

Otherwise we demostrated HRP molecules conjugated to BSA nanoparticles with used to fluorescence and UV absorbance analysis. FIG. 12 shows fluorescence spectroscopy analysis of quenching of BSA nanoparticles with attach of HRP molecules. 100 μL of HRP conjugated Nano BSA in 2 mL deionized water was excited at 310 nm, and emission was recorded at 621.01 nm. Fluorescence intensity value was measured as 288.422 (above peak). HRP conjugated BSA was excited at 310 nm and emission was recorded at 621.01 nm. Fluorescence intensity value of it was measured as 72.8 (below peak). FIG. 12 shows increasing fluorescence intensity of HRP conjugated BSA nanoparticles with attach of HRP/DAB reaction. It was excited at 310 nm, and emission was recorded at 621.01 nm. Fluorescence intensity value was measured as 155.318 (middle peak).

For DAB-based HRP reaction fistly, 2 mg DAB was dispersed in 5 ml dionized water and then it was filtered with a 0.2 μm pore fitler unit and 200 μL was taken in it. After that 500 μL hydrogen peroxide was added into DAB solution and mixed in the sonicator for 30 min. For tested HRP was correctly conjugated to BSA nanocargoes, 10 μL DAB-H₂O₂ solution was added into 1 mg BSA-EDC/NHS-HRP nanoparticles and brownish reaction product was seen. Then nanoparticles were dispersed in 500 μL dionized water and then half amount of 50 μL DAB solution was added and brownish reaction product was seen again so HRP conjugated BSA process was succeed.

Preparation of Heterobifunctional Protein Having Nanocargoes

The following example describe both BSA and lipase and HRP linked and photosensitive aminoacid monomer labeled polymeric nanocargo preparation and its functionalities. For preparing nano MATyr-BSA-lipase-HRP system first of all, microemulsion media was prepared by dispersing 0.5 g of polyvinyl alcohol in 45 mL deionized water. On the other hand, 2000 ppm of BSA, lipase and HRP and MATyr solutions (0.2 g mL⁻¹) was prepared and mixed. Then, oligomer of ruthenium based hapten was added to mixture and mixed for 10 minute additionally. This mixture was added into 25 mL of polyvinylalcohol microemulsion media. Then as an initiator solution, 25 mL of APS solution which was prepared by dissolving 0.02 g APS in 45 ml deionized water added into reaction media. Finally 0.3 ml EDMA was added to mixture and mixed under nitrogen atmosphere during 48 h at room temperature and daylight. After 48 h. occured nanoparticles were seperated from reaction media by centrifugation during 10 minute at 6000 rpm. Nanoparticles was washed with % 50 (v/v) DMSO/water mixture to remove unreacted soluble substances. Then, stored at 0° C. until use.

The activity of the heterobifunctional having BSAnanocargoes were analyzed spectrophotometrically measuring the increment in the absorption at 410 nm promoted by the hydrolysis of pNPP (Winkler and Stuckmann, 1979). For this purpose, firstly stock solution of subtrate containing 20 mM p-nitrophenyl palmitate (p-NPP) in isopropanol was prepared. Then, working substrate was prepared by diluting the p-NPP stock solution (1:20) using 20 mM Tris HCl buffer (pH 8) containing 0.1 mL Triton X-100 and activity of heterobifunctional BSA nanoparticles were measured by mixing 0.9 mL of working substrate and 60 μL of suspended nanoparticles in water. Activity of heterobifunctional nano BSA was found 9.5×10⁻³ μmol paranitrophenol (product)/min (International Unit (IU)).

For tested HRP was correctly conjugated to heterobifunctional BSA nano cargo, nanoparticles was dispersed in 500 μL dionized water and then half amount of 50 μL DAB/H₂O₂ solution was added (in describe 110) and brownish reaction product was seen again by light microscopy so heterobifunctional (lipase and HRP having) BSA nano cargo was succeed.

Synthesis of Nano Transferrin

Nano transferrin particles was prepared by microemulsion polymerization technique by dispersing 0.5 g polyvinyl alcohol in 45 mL deionized water. 200 ppm transferin solution was prepared by dissolving transferin in pH 7 phosphate buffer. Then, 100 μL of 0.2-1.0 g mL⁻¹ MATyr-Ru(bipyr)₂-MATyr complex was added into 200 ppm transferin solution and mixed for 2 h. This solution was added into 25 mL of dispersed polyvinyl alcohol media. On the other hand, APS solution was prepared by 0.02 g APS in 45 mL deionized water as an initiator solution. 20 mL of this initiator solution was added in reaction solution and mixed for 24 h under nitrogen atmosphere at room temperature at daylight. Transferrin nanoparticles was separated from the reaction solution by centrifugation at 6000 rpm for 20 minutes and washed with deionized water to remove unreacted soluble particles.

Affinity Interaction of Nanotransferrin by Antitransferrin Protein Solutions through fluorescence analyses 16 ppm nanotransferrin solution in [83] was prepared in deionized water. Then, 16 ppm antitransferrin solution was prepared in deionized water and it was mixed nanotransferrin solution. FIG. 13 shows fluorescence spectroscopy analysis of 16 ppm nanotransferrin solution, 16 ppm antitransferrin solution and their mixture. Nanotransferrin in 2.0 mL deionized water was excited at 260 nm, and emission was recorded at 521.04 nm. Fluorescence intensity value was measured as 168.390 (below peak). 16 ppm antitransferrin solution was excited at 260 nm, and emission was recorded at 521.04 nm. The fluorescence intensity value was measured as 431.005 (middle peak) and nanotransferrin-antitransferrin mixture was excited at 260 nm, and emission was recorded at 521.04 nm. Fluorescence intensity value was measured as 545.874 (above peak).

Antitransferrin was diluted in different concentration for displaying diluted antitransferin effect to change of fluorescence spectrum. FIG. 14 shows fluorescence spectroscopy analyses of 4 ppm (below peak), 8 ppm (middle peak) and 16 ppm (above peak) antitransferrin solution. Both of them were excited at 260 nm, and emission was recorded at 521.04 nm.

16 ppm of the transferrin solution was prepared to interact with antitransferrin for comparing nanotransferrin-antitransferrin interaction and transferin-antitransferrin interaction. FIG. 15 shows fluorescence spectroscopy analysis of 16 ppm transferrin solution, 16 ppm antitransferrin solution and their mixture. Both of them were excited at 260 nm, and emission was recorded at 521.04 nm. Fluorescence intensity value of transferin was measured as 156.235 nm (below peak). Fluorescence intensity value of antitransferrin was measured as 431.005 nm (middle peak) and fluorescence intensity value of transferin-antitransferrin mixture was measured as 807.889 nm (above peak).

Fluorescence spectroscopy analysis of transferrin-antitransferrin mixture and nanotransferrin-antitransferrin mixture were compared. FIG. 16 shows fluorescence spectroscopy analysis of transferrin-antitransferrin mixture (above peak) and nanotransferrin-antitransferrin mixture (below peak). Both of them were excited at 260 nm, and emission was recorded at 521.04 nm.

Methotrexate Conjugated Nanotransferrin Affect for Cancer Cells

0.06 g matrix media of nanotransferrin particles in [83] were dispersed in 500 μl deionized water and then 100 μl 0.1 M NHS and 100 μl 0.4 M EDC were added into it and mixture was mixed for 2 h. 1 μg methotrexate was dissolved in DMSO and deionized water mixture (5:1) and it was added into mixture and they were mixed for 24 h. Afterwards, solution was centrifuged and sediment was washed with deionized water for three times. For interacting with 5RP7 cancer cells, half of the amount of sediment was dispersed in 100 μL phosphate buffered saline (PBS) and 25 μL DMSO and all of the solution was added into cell culture test plate that had 50 000 5RP7 cells/mL. 25 μL DMSO was added to be DMSO control in another section of cell culture test plate that had 50 000 5RP7cells/mL, too. Also 50 000 5RP7 cells/mL were added into another section of cell culture test plate without using any substance to be control. Flow cytometry analysis of control cells Q1 ratio (necrotic cells) was 0.4%, Q2 ratio (late apoptotic/dead cells) was 0.6%, Q3 ratio (viable cells) was 98.9% and Q4 ratio (early apoptotic) was 0.1%. Flow cytometry analysis of DMSO control cells Q1 ratio was 3.9%, Q2 ratio was 4.3%, Q3 ratio was 91.3% and Q4 ratio was 0.5%. That result demostrated viable cells ratio was still over 90% at DMSO control. 25 μL DMSO was not toxic in that cell culture condition. Cells were stained with Annexin V-FITC and PI Apoptotic Detection Kit for flow cytometry analysis. FIG. 17 showed necrotic cells ratio (PI positive Annexin V negative presented in the left-upper quadrant (Q1) was 3.5%, early apoptotic cells ratio (Annexin V positive PI negative in the right-lower of the FIG. 17 (Q4) and late apoptotic cells ratio (Annexin V and PI positive in the right-upper quadrant (Q2) were respectively 4.2% and 29.6% and viable cells ratio (Annexin V and PI negative in the left-lower quadrant (Q3) was 62.7%. In this manner FIG. 17 was demostrated these specific targeted nanotransferrin approach was killed the cancer cells and viable cells ratio was decreased.

Synthesis of Nano TNF-α and Its Affect for Cancer Cell

Nano TNF-α was prepared by microemulsion polymerization technique. Microemulsion system was prepared by dispersing 0.5 g polyvinyl alcohol in 45 m viable water. 25 mL of this mixture was used for synthesis. 1000 ppm TNF-α solution was prepared in pH 7.0 PBS buffer. 40 μL of 0.2-1.0 g mL⁻¹ MACys was added into TNF-α solution and that were stirred together for 4 h. TNF-α and MACys were added directly into PVA dispersion media. Then 50 μL of 1.0 mg mL⁻¹ aqueous solution of P(MATrp-Ru(bipyr)₂-MATyr) monomer hapten was added into the mixture. On the other hand, initiator solution was prepared by dissolving 0.02 g of ammonium persulfate in 45 mL water. And 20 mL of this initiator solution was added into reaction media and mixed for 36 h at room temperature at daylight under nitrogen atmosphere. Nanoparticles were seperated from reaction media by centrifugation after mixing 20 h and washed with deionized water for three times to remove unreacted materials from nanoparticles occured. Nanoparticles were stored at 0° C. until use.

0.05 g matrix media of nano was dispersed in 1 mL DMSO and 25 μL solution was taken in it and added into cell culture test plate that had 50 000 5RP7 cells/mL. 25 μL DMSO was added into another section of cell culture test plate that had 50 000 5RP7 cells/ml to be DMSO control. Also 50 000 5RP7 cells/ml were added into another section of cell culture test plate without using any substance to be control. 25 μl DMSO was not toxic in that cell culture condition. 5RP7 cells were stained with Annexin V-FITC and PI Apoptotic Detection Kit for flow cytometry analysis. FIG. 18 showed necrotic cells ratio (presented in the left-upper quadrant (Q1) was 8.5%, early apoptotic cells ratio (in the right-lower of the FIG. 18 (Q4) and late apoptotic cells ratio (in the right-upper quadrant (Q2) were respectively 0.6% and 21.0% and viable cells ratio (in the left-lower quadrant (Q3) was 69.8%. In this manner FIG. 18 was demostrated these nanoTNF-α approach was killed the cancer cells and viable cells ratio was decreased.

Preparation of IgG Nanocarrier and Synergic Effect of Phosensitive Ruthenium Based Aminoacid Monomer Hapten Molecule

NanoIgG carrier with photosensitive ruthenium hapten also be usefull in fluorescence detection and in tracking involves various bio-interactions. Photosensitive ruthenium based aminoacid monomer hapten can be used in preparation of nanocarriers through photosensitive polymerization and conjugation, and in detection and imaging of nanocarriers and its interaction.

Nano Immunoglobulin G (IgG) was prepared by microemulsion polymerization technique. Microemulsion system was prepared by dispersing 0.5 g polyvinyl alcohol in 45 mL water. 25 mL of this mixture was used for synthesis. 2 mg of IgG was dissolved in 2 mL deionized water. 40 μL of 1.0 mgmL⁻¹ MATyr-Ru(bipyr)₂-MATrp complex was added to IgG solution and stirred together for 4 h. IgG and MATyr-Ru(bipyr)₂-MATrp complex was added directly to PVA dispersion media. On the other hand, initiator solution was prepared by dissolving 0.02 g of ammonium persulfate in 45 mL water. And 20 mL of this initiator solution was added to reaction media and mixed for 24 h at room temperature at daylight under nitrogen atmosphere. Nanoparticles was seperated from reaction media by centrifugation and washed with deionized water three times to remove unreacted materials from nanoparticles occured. Nanoparticles was stored at 0° C. until use.

Visibility of Nano IgG by Horseradish Peroxidase by Light Microscopy

The most extensive assay application of antibody conjugation using conjugation reagents is for the preparation of antibody-enzyme conjugates. The antibody-enzyme conjugate assay system can be just as sensitive as a radiolabeled antibody system. HRP is one of most popular enzymes used in enzyme-limked immunosorbent assay (ELISA) technology. In the example of the invention will be prepared reusable HRP conjugated nanoantibody carriers.

IgG nanoparticles were dispersed in 500 μL deionized water. 250 μL 0.4 M EDC solution was added into the IgG solution and that was mixed for 1 h on magnetic mixture. After that 250 μL MATyr solution was added and all that mixed for 24 h. The next day 200 ppm 500 μL horse radish peroxidase (HRP) solution was prepared and 250 μL 100 ppm APS, 250 μL of 2.0 mgmL⁻¹ oligomerized polymer hapten of MACys-Ru(bipyr)₂-Cl monomer and 500 μL HRP solution were added to mixture simultaneously. All of them was mixed for 24 h. And light microscopy image of HRP conjugated nano IgG particles shift brownish after DAB/H₂O₂ reaction.

Gold-Protein A Interaction with Nano IgG for Staphylococcal Targeting

500 μL of pH 7.4 phosphate buffer solution was added to IgG nanoparticles. Then, 5 μL gold-protein A was added into 45 μL IgG nanoparticles and solution was mixed for a few minute. FIG. 19 shows quenching of IgG nanoparticles emission with interaction of gold protein A molecules. 500 μL of Nano IgG in 2.5 mL pH 7.4 phosphate buffer solution was excited at 290 nm, and emission was recorded at 581.02 nm. Fluorescence intensity value was measured as 95.455 (below peak). 500 μL of Gold-protein A conjugated nano IgG in 2.5 mL pH 7.4 phosphate buffer solution was excited at 290 nm and emission was recorded at 346.00 and 581.02 nm. Fluorescence intensity value was measured as 70.534 and 128.860 nm (above peak).

In addition, we were demostrated differences of between nano IgG interaction gold-protein A and IgG interaction gold-protein A (FIG. 23). For this purpose above mentioned procedure was repeated with 0.0003 g IgG. 500 μL of IgG conjugated gold-protein A in 2.5 mL pH 7.4 phosphate buffer solution was excited at 290 nm, and emission was recorded at 335.07 and 581.02 nm. Fluorescence intensity value was measured as 374.422 and 243.830. This results shows that nano IgG is effective as IgG and we believe that difference in concentration caused by the difference between emission nano IgG and IgG.

Multi Binding Sites of Gold-Protein A with Nano IgG

Protein A is a 40-60 kDa surface protein originally found in the cell wall of the bacteria Staphylococcus aureus. Protein A is a wall-anchored protein with either four or five domains that each bind to the Fc region of IgG [Timothy J. Foster, IMMUNE EVASION BY STAPHYLOCOCCI, Nature Publishing Group, (2005):948: 3]

In this example we want to prove multi binding sites of gold protein A and especially binding activity of all attaching molecules.

First phase: The example of “Gold-Protein A Interaction with Nano IgG for Staphylococcal Targeting” was repeated.

Second phase: Anti IgG Antibody Interaction with Nano IgG conjugated Gold-Protein A We want to prove active fab region of nano IgG, in this reason we interact anti IgG antibody with Nano IgG conjugated gold-protein A. 0.0003 g anti IgG antibody was dissolved in 500 μL pH:7.4 phosphate buffer solution. 50 μL anti IgG antibody solution was added into the 50 μL nano IgG conjugated Gold-Protein A and the solution was mixed for a few minute. FIG. 20 shows fluorescence spectroscopy analysis of anti IgG antibody interaction with nano IgG conjugated gold-protein A emission. 500 μL Anti IgG antibody interaction with nano IgG conjugated gold-protein A in 2.5 mL pH 7.4 phosphate buffer solution was excited at 290 nm, and emission was recorded at 340.00 and 581.02 nm. Fluorescence intensity value was measured as 216.235 and 250.411 (above peak) and peak at 346.00 nm shows increasing and shifting to 340.00 nm. And peak at 581.02 nm shows increasing as a result of anti IgG interaction with nano IgG conjugated gold protein A (FIG. 23).

Third Phase: Anti IgM Antibody Interaction With [Anti IgG-Nano IgG] Conjugated Gold-Protein A

This example was demostrated multi binding sites region of gold-protein A, firstly anti IgM antibody was interacted with [anti IgG antibody-nano IgG] conjugated gold-protein A. 0.0006 g anti IgM antibody was dissolved in 100 μL pH:7.4 phosphate buffer solution. 50 μL anti IgM antibody solution was added into the 100 μL [anti IgG antibody-nano IgG] conjugated Gold-Protein A. The solution was mixed for a few minute. FIG. 21 shows fluorescence spectroscopy analysis of anti IgM antibody interaction with [anti IgG antibody-nano IgG conjugated gold-protein A emission. 500 μL Anti IgM antibody interaction with [anti IgG antibody-nano IgG conjugated Gold-Protein A in 2.5 mL pH 7.4 phosphate buffer solution was excited at 290 nm, and emission was recorded at 338.00 and 581.02 nm. Fluorescence intensity value was measured as 250.723 and 151.840 (below peak in second spectrum) and peak at the 340.00 nm shows increasing and schifting to 338.00 nm. And peak at the 581.02 nm shows quenching through surface of nano IgG conjugated gold protein A after anti IgM interaction (FIG. 23).

Fourth Phase: IgM Interaction With Anti IgM Antibody Decorated and [Anti IgG Antibody-Nano IgG Conjugated Au-Protein A

0.0006 g IgM was dissolved in 100 μL pH:7.4 phosphate buffer solution. 50 μL IgM was added into the 150 μL anti IgM antibody decorated and [anti IgG antibody-nano IgG conjugated gold-protein A solution and mixed for a few minute. FIG. 22 shows fluorescence spectroscopy analysis of 500 μL IgM interaction with anti IgM antibody decorated and [anti IgG antibody-nano IgG]conjugated gold-protein A in 2.5 mL pH 7.4 phosphate buffer solution was excited at 290 nm, and emission was recorded at 336.00 and 581.02 nm. Fluorescence intensity value was measured as 249.635 and 408.166 (above peak) at the 581.02 nm. And peak at 581.02 nm shows increasing as a result of IgM interaction with anti IgM antibody conjugated onto [anti IgG antibody-nano IgG]conjugated gold-protein A complex (FIG. 23). This peak shows increasing of nanoIgG that both anti IgM and anti IgG antibody conjugated to gold protein A after IgM interaction.

Nano Lipase Preparation

Nano lipase was synthesised according to microemulsion polymerization technique procedure. Microemulsion system was prepared by dispersing 0.5 g PVA in 45 mL deionized water. On the other hand, 0.5 g MATrp was dissolved in 5 mL of dimethylsulfoxide and added into lipase solution prepared by dissolving 10 mg of lipase in 5 mL deionized water. Then, 250 μL of 1.0 mg mL⁻¹ of aqueous solution of polymer of MACys-Ru(bipyr)₂-Cl ruthenium based monomer hapten was added into the mixture and mixed for 1 h. This mixture was added into 25 mL of PVA dispersing medium. Beside this, 20 mL of initiator solution prepared by dissolving 0.002 g APS in 45 mL deionized water was added into reaction mixture. Finally, 0.3 mL of EDMA was added and mixed for 48 h under nitrogen atmosphere at room temperature, at daylight. Lipase nanoparticles was separated from the reaction solution by centrifugation at 6000 rpm for 20 minutes and washed with deionized water to remove unreacted substances.

The nanolipase particles are spherical and between 40-140 nm in diameter (FIG. 24).

Enzyme Activity Assay of Nano Lipase

The activities of free lipase and nanoparticles were analyzed spectrophotometrically measuring the increment in the absorption at 410 nm promoted by the hydrolysis of pNPP (Winkler and Stuckmann, 1979). For this purpose, firstly stock solution of subtrate containing 20 mM p-nitrophenyl palmitate (p-NPP) in isopropanol was prepared. Then, working substrate was prepared by diluting the p-NPP stock solution (1:20) using 20 mM Tris HCl buffer (pH 8) containing 0.1 mL Triton X-100 and activity of lipase and nanoparticles were measured by mixing 0.9 mL of working substrate and 0.1 mL of diluted enzyme sample (or 25 mg nanoparticles).

The pH is one of the important parameters cabaple of alterning enzymatic activities in aqueous solution. In this study, the optimum pH was determined applying different pH values ranging from 6.0-10.0 using 20 mM p-nitrophenylpalmitate. The effect of pH on the time course of p-nitrophenylpalmitate hydrolysis is shown in FIG. 25. It can be seen from figure, with increasing pH (to pH=8.0) the hydrolytic activity was increased both in the presence of free lipase and lipase nanoparticles.

To determine the optimum temperature on p-nitrophenylpalmitate hydrolysis were applied different temperature values ranging from 20° C.-50° C. As can be seen from the FIG. 26, the optimum temperature is approximately 40° C.

The kinetic-constants of p-NPP hydrolysis were obtained fitting the data equation 1, where V is the initial rate, Vm is the maximum rate, S is the concentration of substrate, Km is the Michealis-Menten constant.

V=Vm.S/(Km+S) (1)

Hydrolytic activity of nanoparticles including lipase was evaluated in the framework of

Michealis-Menten kinetics. For this purpose, paranitrophenyl palmitate substrate concentrations in the range of 2 mM-20 mM were used in each case and initial reaction rates (IRR) of hydrolysis were determined. Then, 1/IRR versus reciprocal of the substrate concentration (1/S) for nanoparticles was plotted that is called Lineweaver-Burk plot and the values of Vm and Km were obtained from plots as 0.72 mMmin⁻¹ and 9.3 mM, respectively.

In Michealis-Menten kinetics, Km reflects the affinity of an enzyme for a particular substrate (the lower value of Km the higher affinity). In the present case Km represents the affinity of functional groups of nanoparticles including lipase for substrate p-nitrophenyl palmitate.

In order to show the stability and reusability of the lipase nanoparticles, the experimental cycle was repeated 5 times using the same particles. For sterilization after one cycle, the particles were washed with ethanol. After this procedure, particles were washed with distilled water.

Reusability cycles could be repeated during at least 5 cycles without detecting and significant change in the enzyme activity. After 5 cycles, the enzymatic activity had decreased by ca. 5%.

Synthesis of Nano Lysozyme

Nano lysozyme was prepared by microemulsion polymerization technique. Microemulsion polymerization system was prepared by dispersing 0.5 g polyvinyl alcohol in 45 mL deionized water. Only 25 mL of this dispersed solution was used in nano lysozyme synthesis. On the other hand, 0.5 g MATyr was dissolved in 5 mL of dimethylsulfoxide and added into lysozyme solution prepared by dissolving 10 mg of lysozyme in 5 mL deionized water. Then, 200 μL of 1.0 mg mL⁻¹ aqueous solution of polymer of ruthenium based hapten (MATrp-Ru(bipyr)₂-Cl) was added into the mixture and mixed for 1 h. This prepared mixture was added into PVA dispersing media. On the other hand; 20 mL of APS solution, initiator solution, prepared by dissolving 0.0198 g APS in 45 mL water was added into reaction mixture. Finally, 0.3 mL of EDMA was added and mixed for 48 h under nitrogen atmosphere at room temperature, at daylight. Lysozyme nanoparticles were separated from the reaction solution by centrifugation at 6000 rpm for 20 minutes and washed with deionized water to remove unreacted soluble particles.

| | Colony number | | Colony number | |
|---|---|---|---|---|
| | of S. aureus | Decrease | of E. coli | Decrease |
| Type of sample | (cfu mL⁻¹) | (%) | (cfu mL⁻¹) | (%) |
| Original culture | 154 × 10⁵ | | 526 × 10⁵ | |
| Interacted with | 45 × 10⁵ | 71 | 508 × 10⁵ | 4 |
| Nano lysozyme | | | | |

Nano lysozyme was interacted with S. aureus and E. coli bacteria culture to test its antibacterial activity. For S. aureus bacteria, colony number in the culture was 154×10⁵ before interacted with nanolysozyme particles. After interaction with nanolysozyme particles, colony number of S. aureus bacteria was decreased and it was seen that 45×10⁵. total decrease is % 71 for S. aureus and nanolysozyme was shown an antibacterial effect on S. aureus bacteria.

On the other hand, antibacterial effect of nanolysozyme was studied on E. coli bacteria colony. Colony number of original culture of E. coli was 526×10⁵ before the interaction with nanolysozyme. However, after interaction there was no significant decrease on colony number of E. Coli and it was seen that colony number of E. Coli was 508×10⁵. Only %4 decrease was happened after interaction nanolysozyme with E. Coli bacteria.

Synthesis of Nano Concanavalin A and Its Florescein Isothiocyanate Conjugate

Lectins like conconavalin can be used as targeting molecules to localize particular glycoconjugates and as detecting molecules.

Nano Concanavalin A (ConA) was synthesized acoording to same procedure of microemulsion polymerization technique. First of all, 0.5 g polyvinyl alcohol was dispersed in 45 mL of water to create microemulsion polymerization media. 2 mg of Con A was dissolved in 2 mL of deionized water and 0.1 g mL⁻¹ of MATrp was added into this Con A solution. They are mixed for 2 h. Then, 150 μL of 1.0 mg mL⁻¹ aqueous solution of P(MUABT-Ru(bipyr)₂-MATrp) was added into the mixture. When they are mixing, initiator solution was prepared by dissolving 0.02 g of ammonium peroxodisulfate in 45 ml of water. 25 mL of this solution was added to reaction medium. All the reactants were mixed for 48 h at room temperature at daylight under nitrogen atmosphere. At the end of mixing period, nanoparticles was centrifuged and washed 4 times with deionized water to remove unreacted substances. Nano ConA particles was stored at 0° C. until use.

For preparation of FTIC conjugated nano concanavalin A: 200 ppm, 1 m° C. of NanoCon A dispersion was prepared by deionized water. 150 μL 0.1 M of NHS and 150 μL 0.4 M of EDC were added into 200 ppm Nano Con A dispersion and stirred. On the other hand, fluorescein isothiocyanate solution was prepared by dissolving 0.0002 g fluorescein isothiocyanate (FTIC) in 1.5 mL of deionized water. 500 μL of this solution was added into NanoConA-NHS-EDC system and mixed all of them during 6 h. At the end of mixing period, reaction solution was centrifuged and precipitate was washed with deionized water until removing all unreacted fluorescein from the supernatant.

For fluorescence analyses of FTIC conjugated Nano Con A emission and Nano Con A: 500 μL FTIC conjugated Nano Con A in 1 mL deionized water was excited at 250 nm, and emission was recorded at 500.00 nm. Fluorescence intensity value was measured as 995 and peak at 500.00 nm shows increasing from 790 to 995. And peak at 500.00 nm shows increasing as a result of FTIC conjugaton on to Nano Con A.

Mannose Recognition by Nano Concanavalin A

Mannose is a sugar monomer of the hexose series of carbohydrates and presents in numerous glycoconjugates including N-linked glycosylation of proteins. C-mannosylation is also abundant and can be found in collage-like regions. D-Mannose is a natural occurring simple sugar that appears to be a safe, practical alternative for the treatment of urinary tract infections (UTI's). UTIs start when Escherichia coli (E. Coli) invade the bladder and penetrate a protective coating of the superficial cells that line the bladder. In most cases, urine flow washes out bacteria from the bladder. But the cell wall of E. coli bacteria has tiny finger-like projections that contain complex molecules called lectins on their surface. These lectins are cellular glue that binds the bacteria to the bladder wall so they cannot be easily rinsed out by urination. The chemical structure of D-Mannose causes it to stick to E. coli bacteria, may be even more tenaciously than E. coli adheres to human cells. Although the mechanism of how it works is complicated, theoretically, if enough D-mannose is present in the urine, it binds to the bacteria and prevents them from attaching to the urinary tract lining (Bouckaert et al., 2005; Ofek et al., 1982). D-mannose has been shown to reduce bacteria in rats in a dose dependent manner. In fact, D-mannose was found to significantly reduce bacteria in one day (Michaels et al., 1983). Normal urination, therefore, with a sufficient level of D-mannose present, becomes a simple and effective treatment for treating and preventing UTIs.

In this study, nano-Con A was prepared for recognition of mannose. Mannose detection measurements were performed by fluorescence spectrophotometer (FIG. 27). The mannose adsorption increased with the mannose intial concentration and reached a plateau (at around 5 ppm of mannose concentration at 320 a.u of fluorescein intensity), at which we may assumed that all the active points available for mannose adsorption were occupied with mannose molecules.

IgM Recognition by Nano Concanavalin A

Immunoglobulin M (IgM) plays a central role in the initial response of the immune system to foreign antigens. The serological diagnosis of many human infections is based on the determination of rising pathogen IgM titers during their early appearance (Nigro G, Mattia S, Midulla M., Serodiagn Immunother Infect Dis 1989; 3:355-61.; Punnarugsa V, Mungmee V., J Clin Microbiol 1991:2209-12.; Chen P-J, Wang J, Hwang L, Yang Y, Hsieh C, Kau J, et al. Proc Natl Acad Sci USA 1992; 89:5971-5). IgM content in human serum can be used to estimate immune function and is an important parameter for diagnosing acute and chronic hepatitis, rheumatoid arthritis, hepatocirrhosis, and malignant plasma cell tumors. Thus, a sensitive estimate of IgM is important in clinical laboratories.

IgM is a pentameric molecule in which each monomer consists of 14 immunoglobulin fold domains containing 5-Asn-linked oligosaccharide sites located on the μ heavy chain at residues 170, 332, 395, 402 and 563. The carbohydrate composition of human IgM shows a high mannose content, being totally different from other serumimmunoglobulins. Because of this, mannose binding proteins (MBPs) (For example Con A) have been used for purification of immunoglobulin M (IgM) (Koppel R.; Solomon B.'Journal of Biochemical and Biophysical Methods, Volume 49, Number 1, 30 Oct. 2001, pp. 641-647 (7)).

In this study, nano-Con A was prepared for recognition of IgM. IgM detection measurements were performed by fluorescence spectrophotometer (FIG. 28). As seen from the FIG. 28, the specific IgM adsorption increased with the IgM intial concentration and reached a plateau (at around 5 ppm of IgM concentration), at which we may assumed that all the active points available for IgM adsorption were occupied with IgM molecules.

Synthesis of Nano Avidin and Its Interaction with Biotin Solution

1. Nano avidin was prepared by microemulsion polymerization technique. Microemulsion system was prepared by dispersing 0.5 g polyvinyl alcohol in 45 mL deionized water. 25 mL of this mixture was used for synthesis. 2 mg of avidin was dissolved in 2 mL pH 7.0 phosphate buffer to prepare avidin solution. 40 μL MATyr-Ru(bipyr)₂-Cl complex was added to avidin solution and stirred together for 4 h. Avidin-MATyr-Ru(bipyr)₂-Cl complex was added directly to PVA dispersion media. Then, 50 μL of 1.0 mg mL⁻¹ of aqueous soltion of P(MUABt-Ru(bipyr)₂-MATrp) was added into the mixture. On the other hand, initiator solution was prepared by dissolving 0.02 g of ammonium persulfate in 45 mL deionized water. 20 mL of this initiator solution was added to reaction media and mixed for 48 h at room temperature at daylight under nitrogen atmosphere. Nanoparticles were seperated from reaction media by centrifugation during 12 h and washed with deionized water three times to remove unreacted materials from nanoparticles occured. Nano avidin was stored at 0-25° C. until use.

2. For fluorescence measurement firstly 0.2 g nano avidin nanoparticles were taken and dispersed with 4 mL phosphate buffer. Then, 0.5 mL solution was taken from this stock solution and diluted with 2 mL phosphate buffer again and excited at 310 nm. Then, 0.5 mL stock solution was taken and 2 mL 0.1 ppm biotin was added onto this solution and mixed 2 hour at 20° C., finally this solution also exited at 310 nm. FIG. 29 shows the emission changing of nano-avidin interaction with biotin at 620 nm.

Synthesis of Antitubulin Antibody Nanoparticles and Its Interaction with Tubulin in Cell Fraction

1. Antitubulin antibody nanoparticles were synthesised in micromemulsion polymerisation reaction media. 0.5 g polyvinyl alcohol dispersed in 45 mL deionized water to create the microemulsion polymerisation system. On the other hand, 1 mg antitubulin antibody was dissolved in 1 mL of pH 7 phosphate buffer solution. Beside this, MUABt-Ru(bipyr)₂-MATrp complex solution was prepared by dissolving 0.0008 g of MUABt-Ru(bipyridyl)₂-MATrp in 800 μL methanol. This complex solution was mixed antitubulin antibody during 1 h, then, added into 25 mL of dispersed polyvinyl alcohol dispersed media. After that, 0.0198 g APS in 45 mL deionized water as an initiator solution. 20 mL of this initiator solution was added into reaction mixture and mixed for 48 h under nitrogen atmosphere at daylight and room temperature. Antitubulin antibody nanoparticles were separated from the reaction solution by centrifugation and then washed with deionized water for 3 times to remove unreacted soluble substances.

2. For fluorescence measurement firstly 0.5 mg antitubulin antibody nanoparticles were taken and dispersed with 5 mL phosphate buffer. Then, 1 mL solution was taken from this stock solution and diluted with 2 mL phosphate buffer again and excited at 325 nm. Then, 1 mL stock solution was taken and 2 mL cell fraction was added onto this solution and mixed 3 hour at 0° C., finally this solution also exited at 325 nm. FIG. 30 shows the emission changing of nano-antitubulin particles interaction with cell fraction at 650 nm.

Protein Determination in Nano Protein Particles

Bradford Assay is a rapid and accurate method commonly used to determine the total protein concentration of a sample. The assay is based on the observation that the absorbance maximum for an acidic solution of Coomassie Brilliant Blue G-250 shifts from 465 nm to 595 nm when binding to protein occurs. Both hydrophobic and ionic interactions stabilize the anionic form of the dye, causing a visible color change. Within the linear range of the assay (˜5-25 mcgmL⁻¹), the more protein present, the more Coomassie binds

(Bradford, M. A rapid and sensitive method for the quantitation of microgram quantities of protein utilizing the principle of protein-dye binding, Anal. Biochem. (1976) 72, 248-254).

The amount of proteins in the nanostructures that were prepared using different photosensitive aminoacid monomer and monomer hapten and having different conjugates were determined by measuring the initial and final concentrations of protein within the adsorption medium using Bradford Assay. A calibration curve was constructed with protein solution of known protein concentration (0.02-0.25 mg mL⁻¹) and was used in the calculation of protein amount.

| | | | Protein |
|---|---|---|---|
| | | | Concentration |
| | | Initial Protein | in the |
| | | Concentration | Nanostructures |
| Protein | Nanoprotein | (mgmL⁻¹) | (mgmL⁻¹) |
| Bovin Serum | Nano-BSA | 2.0 | 1.76 |
| Albumin | Nano-BSA-MATyr | 2.0 | 1.89 |
| | Nano-BSA (MATyr- | 2.0 | 1.85 |
| | Ru(bipyr)₂-MATyr) | | |
| | Nano-BSA (FTIC) | 2.0 | 1.88 |
| Lipase | Nano-Lipase (MACys) | 2.0 | 1.97 |
| | Nano-Lipase (MATrp) | 2.0 | 1.97 |
| | Nano-Lipase-MATyr | 2.0 | 1.95 |
| Conconavalin | Nano-Con A | 2.0 | 1.98 |
| A (Con A) | | | |
| IgG | Nano-IgG | 2.0 | 1.99 |
| BSA-HRP- | Nano-BSA-HRP-lipase | 6.3 | 4.65 |
| lipase | | | |

Superoxide dismutase (SOD) separation by anti SOD antibody functionalized superparamagnetic nanoparticles (antiSOD-SPN).

1. 50 mg superparamagnetic nanoparticles (SPN) was dispersed in 1 mL toluen and 0.1 mL 3-(Trimethoxysilyl)propyl methacrylate was added. This solution was mixed 24 h. at 60° C. for silyl activation. The activated SPN was washed several times with ethanol and then phosphate buffer and then dried. To prepare anti SOD antibody functionalized SPN, 200 μL phosphate buffer and 30 μL MATyr-Ru-MATyr were added onto silyl-activated SPN and then 50 μL 200 ppm anti SOD antibody and 100 μL 0.1 mg mL⁻¹ APS were added into this solution and mixed for three hour. The SOD antibody functionalized SPN (anti SOD-SPN) particles were washed several times.

2. For fluorescence measurement firstly 1.0 mg dry anti SOD-SPN was weight and dispersed with 3.0 mL of phosphate buffer (pH 7.0) and measured by fluoremetry (excitation: 275 nm emission: 498.05 nm). Then 1.0 mg anti SOD-SPN was interacted with 3 mL of SOD fraction and measured by fluoremetry. FIG. 31 was shown the quenching of emission by interaction of anti SOD-SPN and SOD fraction. The anti SOD-SPN particles which bind SOD were washed with 0.1 M glycine-HCl. The glycine HCl was removed from particles and lyophilized and so pure SOD was obtained. Finally, enzyme activity of SOD was controlled by a SOD assay method. The method consists of inhibitory SOD capacity of reducing the tetrazolium salt (Nitro Blue Tetrazolium—NBT) at formazans by the superoxide radicals, generated in the reaction medium by riboflavin photoreduction. 1 unit of activity was determined as that amount of enzyme causing half the maximum inhibition of NBT reduction. Activity of the sample was determined as 4.34 U mg⁻¹ prot. at λ=560 nm (Winterbourn C. C., Hawkins R. E., Brain M., Carell R. W.—The estimation of red cell superoxide dismutase activity, J. Lab. Clin. Med., 1975, 85 (2), 337-341).

TNFα-TNFα antibody conjugated SPN interaction and TNFα separation by SPN

1. 50 mg SPN was dispersed in 1 mL toluen and 0.1 mL 3-(Trimethoxysilyl)propyl methacrylate was added. This solution was mixed 24 h. at 60° C. for silyl activation. The activated SPN was washed several times with ethanol and then phosphate buffer and then dried. To prepare anti TNFα antibody functionalized SPN, 30 μL MATyr-Ru-MATyr was added onto Si-activated SPN and then 50 μL 0.5 mg mL⁻¹ anti TNFα antibody and 100 μL 0.1 mg mL⁻¹ APS were added into this solution and mixed for 3 h. The TNFα antibody functionalized SPN was washed several times.

2. For fluorescence measurement firstly 0.5 mg dry TNFα antibody functionalized SPN was weihgt and dispersed with 3.0 mL phosphate buffer (pH 7.0) and measured by fluorimetry. Then, 0.5 mg TNFα antibody functionalized SPN was interacted with 3.0 mL 0.3 ppm, 0.8 ppm, 2.5 ppm and 5 ppm TNFα solutions, respectively and they were measured by fluorimetry. FIG. 32 shows the increasing of emission by increasing of concentration. FIG. 33 shows Scatchart plot of Tnfa rebinding to Tnfα antibody functionalized SPN. As shown in FIG. 33 the affinity constant (Ka) is very high (9.0×10⁷ M⁻¹).

3. The interaction of TNFα-TNFα antibody functionalized SPN was also controlled with UV spectrophotometry by using of Bradford analysis. Beginning TNFα solution and filtrate solution after the interaction with SPN were measured and a decreasing was observed between these two absorbances. TNFα-bound SPNs were washed with 0.1 M Glycine-HCl to obtain TNFα.

Transferrin and Folic Acid Modified Iron Oxide Nanoparticles for Targeting Cancer Cells

Superparamagnetic ironoxide nanoparticles were modified with 3-trimethoxysilyl propyl methacrylate (TSPM). Firstly, 4.12 mM 3-trimethoxysilyl propyl methacrylate was prepared in 5 mL toluene and solution was added onto iron oxide nanoparticles. Mixture was mixed on a magnetic platform for 24 h at 60° C. Then, silanized iron oxide nanoparticles were removed from solution via magnetic separator and then they were interacted with 5 mL MATyr solution. After that iron oxide-TSPM-MATyr solution was dispersed in 5 mL of phospate buffer (pH: 7) and interacted with 5 mL 50 ppm transferrin solutions, APS and 250 μL P(MATyr-Ru(bipyr)₂-Cl solution. Mixture was mixed on a magnetic platform for 24 h.

After transferrin was bound onto silanized ironoxide nanoparticles, folic acid solution was prepared to bind onto it. 0.0022 g folic acid was resolved in 100 mL of deionized water so 0.05 mM folic acid solution was prepared. Then, 500 μl 0.4 M EDC, 500 μL 0.1 M NHS and 1 mL 0.005 mM folic acid solution were added into transferrin modified silanized iron oxide nanoparticles simultaneously and then all of them were mixed on a magnetic platform for 24 h at room temperature and under daylight. Then iron oxide particles were removed from solution via magnetic separator and washed for two times with deionized water. Absorbance of supernatant, washed water and pure folic acid solution were measured with u.v spectrometer to understand amount of folic acid binding to the modified iron oxide nanoparticles. Finally, transferrin and folic acid modified silanized ironoxide nanoparticles were used to analyse of effect for cancer cells (5RP7 cancer cells) by flow cytometry. 5RP7 cells were stained with Annexin V-FITC and PI Apoptotic Detection Kit for flow cytometry analysis. FIG. 34 showed necrotic cells ratio (presented in the left-upper quadrant, Q1) was 39.5%, early apoptotic cells ratio (in the right-lower of the FIG. 34 (Q4) and late apoptotic cells ratio (in the right-upper quadrant (Q2) were 0.8% and 6.7%, respectively and viable cells ratio (in the left-lower quadrant (Q3) was 53%. Also control cells (not interacted with transferrin and folic acid modified silanized iron oxide nanoparticles) were analysed by flow cytometry and Q3 ratio (viable cells) was 95.7%. In this manner FIG. 34 and TEM images (FIG. 35) were demostrated these transferrin-folate mediated approach was killed the cancer cells and viable cells ratio was decreased.

Generation and Functionalization of Quantum Dots with Amino Acid Monomer and Ruthenium Based Aminoacid Monomer Haptens

For CdS synthesis, 0.01M of Cd(OAc)₂.2H₂O solution (24 mL) was prepared with ethanol. Solution was stirred continuously for 30 min in nitrogen ambient. Sodium sulfide (0.01 M, 24 mL) was slowly added, stirred under nitrogen ambient for 30 min and then centrifuged to collect precipitate. It was washed in double distilled water and dried in air. The entire synthesis was carried out at room temperature (Diltemiz, S. E., Say, R., Büyüktiryaki, S., Hür, D., Denizli, A., Ersöz, A., (2008) Talanta 75, 890-896). CdS/QD were excited at 310 nm, and emission was recorded at 620.0 nm. Fluorescence intensity value was measured as 301.9.

a. QD's Functionalization with MACys Monomer

During a day 5 mL CdS nanocrystals were immersed in ethanol containing 0.018 M, 10 mL MACys in order to introduce methacryloyl groups onto the surface of CdS nanocrystals. The nanocrystals were then washed with ethanol and deionized water for 10 min to remove the excess of thiol groups. A stable self-assembled monolayer of MACys was formed onto the nanocrystal surfaces after all these steps. Fluorescence value was measured by 20 times dilution. CdS/QD were excited at 310 nm, and emission was recorded at 621.01 nm. Fluorescence intensity value was measured as 491.981. MACys modified nanoparticle's fluorescence intensity was increased according to CdS.

b. QD's Functionalization with MUABt-Ru(bpy)₂-MATrp Complex

Qunatum dots functionalized with MUABt-Ru(bpy)₂-MATrp complex. 1 mL was taken from CdS solution and added to 1 mL MUABt-Ru(bpy)₂-MATrp solution (5 mg mL⁻¹), diluted with ethanol to 20 mL and mixed during a day. Fluorescence value was measured by 20 times dilution. CdS/QD were excited at 310 nm, and emission was recorded at 621.01 nm. Fluorescence intensity value was measured as 251. MUABt-Ru(bpy)₂-MATrp modified nanoparticle's fluorescence intensity was decreased according to CdS.

c. QD-MACys's Functionalization with MATyr Monomer

MACys modified qunatum dots functionalized with MATyr monomer. 1 mL was taken from CdS-MACys solution and added to MATyr solution which was dissolved in (5.08 mg) 1 mL water, diluted with ethanol to 20 mL and mixed during a day. Fluorescence value was measured by 20 times dilution. CdS/QD were excited at 310 nm, and emission was recorded at 621.01 nm. Fluorescence intensity value was measured as 426.830. MATyr modified nanoparticle's fluorescence intensity was decreased according to CdS-MACys.

d. QD-MACys's Functionalization with MATrp Monomer

MACys modified qunatum dots functionalized with MATrp monomer. 1 mL was taken from CdS-MACys solution and added to MATrp solution which was disolved in (5.78 mg) 1 mL water, diluted with ethanol to 20 mL and mixed during a day. Fluorescence value was measured by 20 times dilution. CdS/QD were excited at 310 nm, and emission was recorded at 621.01 nm. Fluorescence intensity value was measured as 319.620. MATrp modified nanoparticle's fluorescence intensity was decreased according to CdS-MAC.

e. QD-MAC's Functionalization with MATrp-Ru(bpy)₂-MATrp Complex

MACys modified qunatum dots functionalized with MATrp-Ru(bpy)₂-MATrp complex. 1 mL was taken from CdS-MACys solution and added to 1 ml MATrp-Ru(bpy)₂-MATrp solution which was disolved in (5.00 mg) 1 mL of water, diluted with ethanol to 20 mL and mixed during a day. Fluorescence value was measured by 20 times dilution. CdS/QD were excited at 310 nm, and emission was recorded at 621.01 nm. Fluorescence intensity value was measured as 451.960. MATrp-Ru(bpy)₂-MATrp complex modified nanoparticle's fluorescence intensity was decreased according to CdS-MACys.

f. QD-MAC's Functionalization with MUABt-Ru(bpy)₂-MATrp Complex

MACys modified qunatum dots functionalized with MUABt-Ru(bpy)₂-MATrp complex. 1 mL was taken from CdS-MACys solution and added to 1 mL MUABt-Ru(bpy)₂-MATrp solution which was disolved in (5.00 mg) 1 mL of water, diluted with ethanol to 20 mL and mixed during a day. Fluorescence value was measured by 20 times dilution. CdS/QD were excited at 310 nm, and emission was recorded at 622.02 nm. Fluorescence intensity value was measured as 328.062. MUABt-Ru(bpy)₂-MATrp complex modified nanoparticle's fluorescence intensity was decreased according to CdS-MACys.

Preparation of QD-Photosensitive Aminoacid Monomer-Anti TNT Nanobioconjugate and Its TNT Interaction

10 ppm 1 mL anti-TNT solution, 100 μl 1 mg mL⁻¹ photosensitive hapten polymer and 100 μL 100 mM APS solution in pH 7.4 buffer, respectively, were added to 1 mL conjgated nanocrystal (CdS-MACys-photosensitive aminoacid monomer) solution and polymerized under daylight during a day. Nanocrystals centrifuged and re-dispersed pH 7.4 buffer. Fluorescence value was measured by 20 times dilution. CdS/QD were excited at 310 nm, and emission was recorded at 621 nm. When cross-linking conjugation of anti-TNT was occured, fluorescence intensity was increased. And then, 4 ml of 10⁻² ppm, 0.2 ppm, 0.5 ppm, 0.8 ppm and 1.0 ppm TNT solutions were added to 1 ml of anti-TNT conjugated CdS/QD, respectively. The fluorescence intensity values of these interactions were measured in reference to dilution factor. The fluorescence intensity of the anti-TNT conjugated CdS/QD nanoshells can be enhanced by TNT concentration. The enhancement of fluorescence intensity is proportional to TNT concentration.

Preparation of Conconavalin A Conjugated CdS Nanocrystals and Its IgM Interaction

Qunatum dots functionalized with MATyr monomer. 1.0 mL was taken from CdS-MACys solution and added to MATyr solution which was dissolved (5.08 mg) in 1.0 mL of water and mixed during a day. Fluorescence value was measured by 50 times dilution. CdS/QD were excited at 310 nm, and emission was recorded at 620.0 nm. Fluorescence intensity value was measured as 667.989. 5 ppm 1.0 mL of Conconavalin A solution, 100 μl 1 mg mL⁻¹ P[MATyr-Ru(bipyr)₂-MATyr] and 100 μl 100 mM APS solution in pH 7.4 buffer, respectively, were added to 2 mL CdS-MACys-MATyr solution and polymerized under daylight during a day. Nanocrystals were centrifuged and re-dispersed pH 7.4 buffer. Fluorescence value was measured by 50 times dilution. CdS/QD were excited at 310 nm, and emission was recorded at 621.01 nm. Fluorescence intensity value was measured as 134.383. When polimerization was occured, fluorescence intensity was decreased.

Then, these particules were seperated into four parts and 1 ml 10⁻² ppm, 10⁻¹ ppm, 0.2 ppm, 0.5 ppm, 0.8 ppm and 1.0 ppm IgM solutions added to these parts, respectively. The fluorescence intensity values of these interactions were measured in reference to dilution factor. The fluorescence intensity of the Concanavalin A interacted CdS/QD nanoshells can be enhanced by IgM concentration. The enhancement of fluorescence intensity is proportional to IgM concentration.

Creation of QD's which Can Recognize Hepatitis-B Viruse

100 μL (0.5 mg mL⁻¹) anti-Hepatitis B solution, 100 μl 1 mg mL⁻¹ P[MATyr-Ru(bipyr)₂-Cl] and 100 μL 100 mM APS solution in pH 7.4 PBS buffer, respectively, were added to 2 mL CdS-MACys-MATyr solution and polymerized under daylight during a day. Fluorescence value was measured by 50 times dilution. CdS/QD were excited at 310 nm, and emission was recorded at 621.01 nm. Fluorescence intensity value was measured as 414.629. When polymeric shell was occured, fluorescence intensity was decreased. Then, the polymeric shell having QD solution was centrifuged and diluted to 1 mL with pH 7.4 PBS buffer and interacted with 150 μL Hepatite B vaccine. These mixtures mixed for one hours, centrifuged and dispersed to 3 mL with PBS and fluorescence intensity values were measured. Fluorescence intensity value was measured as 285.057. When the Anti Hepatit B conjugated and polymerised shell having QDs interacted with Hepatite B vaccine, fluorescence intensity was decreased.

Orientation and Conjugation Between Two Antibodies Conjugated Nano Structures

1. The interaction between functionalized quantum dots (QDs) and functionalized Au/Ag nanoclusters was investigated using fluorescence spectroscopy. To prepare antiIgG antibody functionalized Au/Ag nanocluster, 1.5 mg MACys activated Au/Ag nanocluster (as explained in Interaction between avidin and biotinylated TNF-α antibody conjugated Au—Ag nanoclusters) were dispersed in 3 mL phosphate buffer and 3 mg MATyr was added this solution and mixed for 3 h. End of this period, 100 μL of ruthenium based hapten oligomer, 0.6 mg anti IgG antibody and finally 100 μL 0.1 mg mL⁻¹ APS were added into this solution and mixed for three hour. To prepare IgG functionalized QDs, 2 mg MACys activated QDs were dispersed in 3 mL phosphate buffer and 3 mg MATyr was added to this solution and mixed for 3 h. End of this period, 100 μL of ruthenium based hapten oligomer, 0.8 mg IgG and finally 100 μL 0.1 mg mL⁻¹ APS were added into this solution and mixed for 3 h. Then the particles were isolated with centrifigation and washed several times with de-ionized water, and alcohol.

2. Obtained QDs and Au/Ag nanoclusters were dispersed in 3 mL phosphate buffer. For fluorescence measurement firstly 0.5 mL IgG conjugated QDs (as explained in Section Generation and Functionalization of Quantum Dots with amino acid monomer and ruthenium based aminoacid monomer haptens) were taken and diluted with again 2.5 mL phosphate buffer and excited at 250 nm and then 0.5 mL anti IgG antibody functionalized QDs were taken and 0.1 mL anti IgG antibody functionalized nanoclusters and 2.4 mL phosphate buffer were added onto QDs. FIG. 36 shows the quenching of emission of QDs by increasing of Au/Ag nanoclusters.

Interaction between Avidin and Biotinylated TNF-α Antibody Conjugated Au—Ag Nanoclusters

1. Au—Ag nanocluster was synthesized according to Ref. (Gultekin A, Diltemiz S E, Ersoz A, et al., TALANTA (2009) 78, 4-5, 1332-1338). 50 mg Au—Ag nanocluster was dispersed in 1 mL toluen and 1.0 mM MACys was added. This solution was mixed 24 h for methacryloyl activation. The activated Au—Ag nanostructure was washed several times with toluen and then dried. To prepare biotinylated TNF-α antibody functionalized Au—Ag nanoparticles, 5.0 mg mL⁻¹, 20 μL MACys-Ru(bipyr)₂-MACys was added onto methacryoyl-activated Au—Ag nanoparticles and then 200 μL, 1.0 mg mL⁻¹ biotinylated TNF-α antibody and 50 μL 100 mM APS were added into this solution and mixed for 3 h. The biotinylated TNF-α antibody functionalized Au—Ag nanoparticles was washed with phosphate buffer several times.

2. For fluorescence measurement firstly; 0.5 mg dry biotinylated TNF-α antibody functionalized Au—Ag nanoparticle was weight and dispersed with 3.0 mL phosphate buffer (pH 7.0) and then 0.5 mg biotinylated TNFα antibody functionalized Au—Ag nanoparticle was interacted with 3.0 mL of 1.16×10⁻⁸ M, 1.74×10⁻⁸M, 2.32×10⁻⁸ M, 2.9×10⁻⁸ M, 3.48×10⁻⁸M, Avidin solutions (in phosphate buffer pH 7.0), respectively. According to Scatchart plot of avidin rebinding to biotinylated TNF-α antibody functionalized Au—Ag nanoparticles. The affinity constant (Ka) is very high (1.98×10⁷ M⁻¹).

Biotin Recognition by Biotin Imprinted Au/Ag Nanoclusters

1. To prepare biotin imprinted Au/Ag nanocluster, 1.5 mg metacrolylamidocistein (MACys) activated Au/Ag nanocluster were dispersed in 5 mL phosphate buffer and modified with 20 μL MATyr-Ru(bipyr)₂-MATrp (5.0 mg mL⁻¹), and then added into cross-linking solution of 200 μL avidin (100 μg mL⁻¹) and 200 μL biotin (400 μg mL⁻¹) preorganized affinity unit, 0.25 mL N,N′-Methylenebisacrylamide (0.1 M) and 50 μL 100 mM APS mixture and then mixed for ten hour at day light. Then the particles were isolated with centrifguation and mixed with 5 mL 0.1 M NaOH for 24 h for removal of biotin template and then washed several times with deionized water, and alcohol. Finally, the biotin imprinted nanostructures were dispersed in 3 mL phosphate buffer and stored.

2. For fluorescence measurement firstly, 50 μL dispersed biotin imprinted nanocluster was taken and mixed with 3.0 mL 8.19×10⁻⁷ M biotin solution. Then 50 μL dispersed biotin imprinted nanoclusters were interacted with 3.0 mL 1.23×10⁻⁶, 1.64×10⁻⁶, 2.05×10⁻⁶, 2.46×10⁻⁶ M biotin solutions, respectively and all measured by fluorimetry. FIG. 37 was shown the increasing of emission by increasing of concentration.

Ambush and Cell Targeting Approach of P450 Gene-Directed Enzyme Prodrug Therapy and Imaging of Cancer Cells

For CdS synthesis, 0.01M of Cd(OAc)₂.2H₂O solution (24 mL) was mixed with ethanol solution and stirred continuously for 30 min in nitrogen ambient. Sodium sulfide (0.01 M, 24 mL) was slowly added, stirred under nitrogen ambient for 30 min and then centrifuged to collect precipitate. It was washed with double distilled water and dried in air. The entire synthesis was carried out at room temperature. After that 5 mL CdS nanocrystals were immersed in ethanol containing 0.018 M, 10 ml MACys in order to introduce methacryloyl groups onto the surface of CdS nanocrystals (as explained in Section Generation and Functionalization of Quantum Dots with amino acid monomer and ruthenium based aminoacid monomer haptens). The nanocrystals were then washed with ethanol and deionized water for 10 min to remove the excess of thiol groups. A stable self-assembled monolayer of MACys was formed onto the nanocrystal surfaces after all these steps. Then 5.08 mg MATyr was dissolved in 1 ml of water. 1 mL was taken from CdS-MACys solution and added to MATyr solution and mixed for 24 h. After that two different ways were applied with these modified quantum dots particles. One of them, 25 μL P450 solution was added to these particles with 50 μL 1 mg mL⁻¹ P[MATyr-Ru(bipyr)₂-Cl] and 50 μL 100 mM APS simultaneously for 24 h at room temperature and daylight. For other one, 50 ppm 500 μL transferrin solution was prepared and added onto modified quantum dots with 50 μL ruthenium based polymer solution and 50 μL APS simultaneously at the same condition like other one. On the following day 50 ppm 500 μl transferrin solution was added with 500 μL 0.1 M NHS, 500 μL 0.4 M EDC and 500 μL MATyr simultaneously onto first solution that contained P450 modified QD. Then 25 μL P450 solution and 500 μL 0.1 M NHS, 500 μL 0.4 M EDC and also 500 μL MATyr were added onto second solution that contained transferrin modified QD. In these way, we could investigate a specifically and synergically targeting and ambush theranostic for cancer cells.

After that Ag—Au particles were modified for prodrug therapy. Firstly, Ag—Au particles were modified with MACys solution (as explained in Interaction between avidin and biotinylated TNF-α antibody conjugated Au—Ag nanoclusters). Then, 1 mL MATyr solution was added onto 1 mL Ag—Au-MACys solution and mixed all of them for 2 h. Later 50 ppm antitransferrin solution was prepared in phosphate buffer and 1 mL antitransferrin solution, 100 μL of ruthenium based polymer solution and 100 μL 100 mM APS were added onto MATyr modified Ag—Au-MACys solution. All of them were mixed on magnetic mixer for 24 h. After 24 h prodrug solution was prepared to add onto solution. Ifosfamide (IFA) was used like a prodrug and 0.007275 g IFA was resolved in 25 μL deionized water. Then, IFA solution, 1 mL of 0.1 M NHS and 1 mL of 0.4 M EDC solutions were added onto antitransferrin conjugated Ag—Au nanostructure and all of them were mixed on magnetic mixer for 24 h.

These ambush and cell targeting based P450 gene-directed enzyme prodrug therapy was tested with human promyelocytic leukemia cells (HL-60) by using flow cytometry. Various experiments were carried out and the best result was observed when IFA conjugated Au—Ag nanoclusters were delivered 4 h later after transferin decorated P450 conjugated QD particles were delivered to the HL60 cells. Flow cyctometry analysis of HL-60 cells stained with annexin V-FITC and PI. This staining distinguishes four subsets of cells: necrotic cells (PI positive), early apoptotic cells (annexin V positive and PI negative), late apoptotic and/or dead cells (annexin V positive and PI positive) and viable cells (no staining). FIG. 38 showed necrotic cells ratio (presented in the left-upper quadrant, Q1) was 0.1%, early apoptotic cells ratio (in the right-lower of the FIG. 38, Q4) and late apoptotic cells ratio (in the right-upper quadrant (Q2) were respectively 34.4% and 15.2% and viable cells ratio (in the left-lower quadrant, Q3) was 50.3%. Also control cells (not interacted with P450 conjugated QD particles and IFA conjugated Au—Ag nanoclusters) were analysed by flow cyctometry and Q3 ratio (viable cells) was 93.2%. In this manner FIG. 38 and FIG. 39 were demostrated these approach was killed the cancer cells and viable cells ratio was decreased.

Preperation of Photosensitive Protein Having Cryogel Using Lysozyme-MATyr and Its Using as Antibacterial Material

Lysozyme-MATyr (Lys-MATyr) doped acrylamide cryogels were prepared by free radical cryo polymerization of monomer solution of Acrylamide with crosslinker N,N′-Methylene bis acrylamide (MBAAm) initiated by N,N,N′,N′-Tetramethyl-ethylenediamine (TEMED) and Ammonium per sulfate (APS) in plastic petri dish. First of all, for preparation of Lys-MAT, 0.026 g MATyr was dissolved in 5 ml deionized water. Then, it was treated ultrasonically for 3 h. On the other hand, the same amount of lysozyme was dissolved in 5 mL of deionized water. 3 mL of each solution were taken and mixed 5 h at 250 rpm at room temrepature. As a general procedure of preparing cryogel matrix, AAm monomer and cross-linker (MBAAm) were dissolved in deionized water with stirring. The monomer concentration was 7% (w/v) with an AAm/MBAAm molar ratio of 10:1. Then, Lys-MATyr mixture (2% (w/v) of the total mass of AAm+MBAAm) added the solution and mixed at 150 rpm during 30 min. Then, solution was cooled for 3-4 min. in an ice bath. TEMED (1% (w/v) of the total mass of AAM+MBAAm) and APS (1.2% (w/v) of the total mass of AAM+MBAAm) were quickly added into the solution as redox initiators. The mixture was stirred during 1-2 min., then poured into petri dish. This cryogel matrix in petri dish was freezed at −18° C. during 16 h. Finally, it was thawed at room temperature. Formed cryogel material was washed with deionized water 3 times to remove unreacted soluble materials. This cryogel material was interacted with S. Aureus and M. Luteus bacteria (Table 1).

TABLE 1
Decreasing of M. Luteus and S. Aureus bacteria after Lys-MATyr
doped acrylamide cryogel interaction

| | Colony | | Colony | |
|---|---|---|---|---|
| | Number of | | Number of | |
| | M. luteus | Decrease | S. aureus | Decrease |
| Type of Sample | (cfu mL⁻¹) | (%) | (cfu mL⁻¹) | (%) |
| Original culture | 182 × 10⁵ | | 1 × 10⁸ | |
| MATyr-Lysozyme | 99 × 10⁵ | 45.6 | 74 × 10⁵ | 92.5 |

After this treatment, antibacterial cryogel material was weight and then devoted to two equal weights in the sterile fresh tubes. 5 mL nutrient broth was added to all tubes. And then one serial of these tubes were inoculated with Escherichia coli from the overnight culture and the other serials were inoculated with S. aureus from the overnight culture. An uninoculated tube (5 m inoculated Nutrient broth) was observed for blank. For the positive control, S. aureus and E. coli were inoculated tubes. After incubation at 37° C. for 24 hours, all tubes were mixed by vortex. Supernatant were taken and measured at 540 nm together negative controls (blank) and positive controls (Absorbance of S. aureus positive control: 2.139 and Absorbance of E. coli positive control: 2.068). Decreases of total cell mass were determined according to changing of turbidities (Table 2). On the other hand, FIG. 40 shows SEM images of (A) Lys-MAT doped acrylamide cryogel and (B) Lys-MAT doped acrylamide cryogel with M. Luteus fixation.

TABLE 2
Interaction of S. Aureus and E. Coli with Lys-MATyr doped
acrylamide cryogel and decreases of total cell mass by changing
of turbitidies spectrophotometrically at 540 nm.

| | Weight of the | | |
|---|---|---|---|
| Materials | materials in tube (g) | S. aureus | E. coli |
| MATyr-Lysozyme cryogel | 0.2 | 2.000 | 1.793 |

Preparation of ruthenium based aminoacid polymer doped silica nanoparticle and its modification by aminoacid monomer (MATyr) and photosensitive conjugation and cross-linking of lysozyme and its Doped Acrylamide Cryogels as Antibacterial Material 1. The W/O microemulsion was prepared by adding 1.8 mL Triton X-100, 7.5 mL cyclohexane, 1.8 mL n-hexanol, and 340 μL of water. 1 mL 1 mg mL⁻¹ P[MATrp-Ru(bpy)₂-MATrp] and 16.7 mL water were added and stirred continuously.

Polymerization reaction was initiated by adding 100 μL TEOS and 60 μL ammonia solution and allowed to continue during a day. After the reaction was completed, photosensitive ruthenium hapten polymer doped silica nanoparticles isolated by acetone and then centrifuged to collect precipitate. It was washed in ethanol and water. The entire synthesis was carried out at room temperature. Ruthenium based aminoacid polymer doped silica nanoparticles were excited at 350.0 nm, and emission was recorded at 701.0 nm. Fluorescence intensity value was measured as 580 nm.

2. Functionalization of Ru doped Si nanoparticles: 5 mg ruthenium based aminoacid polymer doped Si nanoparticles was dispersed 5 mL acetone and added 0.5 mL 3-(trimethoxysilyl)propyl methacrylate. The reaction allowed to continue for 24 h. After the reaction was completed, nanoparticles centrifuged to collect precipitate. Modified Si nanoparticles were excited at 350.0 nm, and emission was recorded at 701.0 nm. Fluorescence intensity value was measured as 454. Second functionalization was carried out with MAT monomer. 5 mL MATyr solution was added to nanoparicles and interacted during a day. When surface modification was occured, fluorescence intensity was decreased.

3. Preparation of a Enzyme Conjugated Silica Nanoparticle: 5.0 mL 30 mg mL⁻¹ lysozym solution was added to functionalized and ruthenium based polymer doped silica (FIG. 41) nanooparticles and added APS. Photosensitive conjugation of lysozyme monomer reaction was carried out under daylight during a day. Nanoparticles were excited at 350.0 nm, and emission was recorded at 701.0 nm. Fluorescence intensity value was measured as 122. When polimerization was occured, fluorescence intensity was decreased.

Functionalization of Carboxylated CNT with MAArginin (MAArg) and Photosensitive and Covalent Conjugation of Annexin V and Its Image Application

For the chemical oxidation and functionalization of carbon nanotubes, 1.0 mg SWNTs were mixed with the solution of concentrated HNO₃ and H₂SO₄ in 1:3 ratio and was ultrasonicated for around 24 h at 70° C. The resultant black mixture was then centrifuged. For the Functionalization of Carboxylated CNT with MAArginin and ruthenium based monomer hapten, first of all, 0.4 M 1 mL of EDC and 0.1 M 1 mL of NHS were interacted. 0.1 mg carboxylated CNT were dispersed with 2 mL of deionized water and mixed with NHS/EDC mixture. Afterward 10 mM 2 mL of MAArginin solution and 0.5 mL of 5.0 mg mL⁻¹ (MATrp-Ru(bipyr)₂-MATrp) was added to this mixture and interacted during a day.

For the Annexin V-FITC modification, MAArg and photosensitive hapten functionalized CNT was dispersed with 1 mL of deionized water and mixed with 10 μL Annexin V-FITC, 20 μL binding buffer and 100 mM 50 μL APS. Then solution was mixed for 24 h. At the end of 24 h solution was centrifuged at 10000 rpm for 10 min and sediment was washed with deionized water. After that sediment was dispersed in 10 mL PBS pH 7.4. Activity of Annexin V-FITC modified CNT was tested with human cheek cells by using fluorescence microscope. The inner lining of human cheek was gently scraped. The cells were transfered into a eppendorf tube that had 50 μL PBS pH 7.3. Then 50 μL Annexin V-FITC modified CNT was added into the eppendorf tube and cells were incubated for 15 min. Afterward 25 μL sample was taken to observe activity of Annexin V-FITC modified CNT with using Leica fluorescence microscope. According to cell images demostrated living cell was not labeled with Annexin V-FITC modified CNT, only apoptotic cells was labeled. In conclusion, Annexin V-FITC modified CNT was active to interacted with phosphatidylserine side on the apoptotic cell surface.

Conjugation and Cross-Linking of Lipase Within Nanolayers of 2-Acrylamido-2-methyl-1-propansulfonicacid Modified Smectite

For the preparation of organosmectite, 20 g of the simectite was dispersed in 500 mL deionized water at 80° C. Then, concentrated 5 mL of HCl in 100 mL deionized water was added, and the solution was heated and stirred for 3 h. The suspension was filtered, and the solid residue was washed with hot distilled water. The product was dried at 55° C. for several days in a fan oven, then dried under vacum for 24 h. And the product was modified in a solution of 0.05 mol 2-Acrilamido-2-methyl-1-propansulfonicacid and yielding the 2-Acrylamido-2-methyl-1-propansulfonicacid modified simectite. Follows 2-Acrylamido-2-methyl-1-propansulfonicacid modified smectite were added to 0.5 mL of 4 mg mL⁻¹ MATyr-Ru(bipyr)₂-MATyr and 10 mL deionized water. Then, 1000 ppm, 10 mL lipase and 40 mg APS were added to the dispersion medium. Reaction mixture was carried out at 25° C. with continuos stirring using magnetic stirrers. All reactions were run for 3 h.

The activity of lipase which is in the nanolayers of organoclay was analyzed spectrophotometrically measuring the increment in the absorption at 410 nm promoted by the hydrolysis of pNPP (Winkler and Stuckmann, 1979). For this purpose, firstly stock solution of subtrate containing 20 mM p-nitrophenyl palmitate (p-NPP) in isopropanol was prepared. Then, working substrate was prepared by diluting the p-NPP stock solution (1:20) using 20 mM Tris HCl buffer (pH 8) containing 0.1 mL Triton X-100 and activity of lipase in the layers of smectite measured by mixing 0.9 mL of working substrate and 60 μL of suspended nanoparticles in water.

Activity of lipase in organoclay nanolayer was found 3.9×10⁻³ μmol paranitrophenol/min (International Unit (IU)).

In order to show the stability and reusability of the lipase conjugated organoclay, the experimental cycle was repeated 5 times using the same particles. For sterilization after one cycle, the particles were washed with ethanol. After this procedure, lipase conjugated organoclay were washed with distilled water. Reusability cycles could be repeated during at least 5 cycles without detecting and significant change in the enzyme activity. After 5 cycles, the enzymatic activity had decreased by ca.7%.

Protein Conjugation on to Quartz Crystal Microbalance Sensor

1. Binding events were followed using a Research Quartz Crystal Microbalance (RQCM) with phaselock oscillator, Kynar crystal holder, 100-4 cell volume flow cell and 1-inch, Ti/Au, AT-cut, 5-MHz quartz crystals (all purchased from Maxtek, Inc). The holder was mounted with crystal face positioned 90° to ground to minimize gravity precipitation onto the surface. A variable-flow minipump (Minipuls 3) peristaltic pump with 0.51 mm PVC tubing (Tygon R 3603) was used with the flow cell at rates in the range of 0.1-0.5 ml min⁻¹. Fresh tubing was cut before each run in order to keep contamination at a minimum level and to limit flow rate deviations. The RQCM phase-lock oscillator provided loading resistance measurements and allowed for the examination of crystal damping resistance during frequency measurements. All measurements were recorded at room temperature. Sensitivity is known to be 56.6 Hz cm² μg⁻¹ for a 5-MHz crystal.

2. Gold surfaces of QCM electrodes were cleaned in a piranha solution (1:3 30% H₂O₂/concentrated H₂SO₄) for 3 min before coating. The cleaned gold surfaces were immersed into MACys solution (10 mM) for 24 h, in order to introduce thiol groups on to gold surface of QCM sensors. The sensors were then washed with ethanol and deionized water for 10 min. to remove the excess of thiols. A stable self-assembled monolayer of thiol was formed on to electrode surfaces after all these steps. For the polymerization, the reaction mixture containing the (MATyr-Ru(bipyr)₂-MATyr) monomer, avidin (100 μg) and APS (2.5 mmol) in HBS buffer was degassed and dropped on QCM sensors. Polymerisation was carried out at room temparature for 4 h.

3. The crystal was mounted in the holder/flow cell, rinsed with pH 7.4 HBS buffer (10 mM HEPES, 150 mM NaCl, 3.4 mM EDTA) and brought to resonant frequency. Biotin was dissolved in HBS buffer (pH 7.4) to have a concentration from 0-40 μg mL⁻¹ and pumped through the flow cell at 0.1 mL min⁻¹. The frequency of the sensor was monitored until it became stable. The frequency shift for each concentration of biotin was determined and the evaluation was performed in triplicate. After each assay, biotin was removed from the coating by washing with 0.1 M glycine-HCl (pH: 1.8) (0.5 mL min⁻¹, 60 min) and then three times with HBS buffer. The frequency of the sensor approximately recovered to the value of beginning resonant frequency.

4. The avidin coated sensor is expected to bind the biotin sensing. The frequency of the sensor decreased after pumping the biotin solution, then reached the constant value in 30 min (FIG. 42). It can be seen that the reaction reached equilibrium quickly. These frequency changes strongly indicated that the biotin molecules were bounded to the polymer on the quartz crystal.

Biotin solutions with different concentration between 0-40 μg mL⁻¹ were pumped to electrode. The obtained frequency shift values showed calibration curve of the sensor (FIG. 43).

Concanavalin A—Sialic Acid Interaction and Sialic Acid determination by Surface Plasmon Resonance (SPR)

1. The interaction between concanavalin A coated sensors and sialic acid was investigated using Surface Plasmon Resonance system. For this purpose, the electrodes were cleaned in a piranha solution for 3 min (1:3 30% H₂O₂/concentrated H₂SO₄). The sensors were then washed with ethanol and deionized water. For the polymerization, the reaction mixture containing the (MUABt-Ru(bipyr)₂-MATrp) monomer, concanavalin A (1 mmol) and APS (2.5 mmol) in HEPES buffer was degassed and dropped on SPR sensors. Polymerization was carried out at room temperature for 4 h. After the polymerization, sensors were washed three times with HEPES buffer and stored at +4° C. in refrigerator until using.

2. The SPR measurement was performed using the following procedure.

The sensors were located in SPR system and HEPES buffer was injected to baseline. For surface plasmon resonance measurement, reaction mixture containing the sialic acid (1 mmol), 0.4 M EDC and 0.1 M NHS was injected with flow rate of 5 μL min⁻¹. Then, the sensor surface was rinsed with HEPES buffer solution and regenerated with 0.1 M glycine-HCl buffer solution (pH: 2.0) for removal of sialic acid, then washed again with water. The adsorption isotherm of sialic acid on polymer coated sensor can be obtained by plotting the observed reflectivity as a function of the time (FIG. 44). As seen in figure reflectivity increased at approximately 15^{th} min. because of interaction between concanavalin A with sialic acid.

1. A method of making a covalent immobilization and photosensitive crosslinking by ruthenium-chelate based monomers of a vast range of proteins on a different monomer linker having micro and nanosurfaces by irradiation, the method comprising the steps of:
synthesizing ruthenium based aminoacid monomers and oligomers as photosensititive metal-chelates;
preparing a monomer linker having surfaces;
reacting a photosensitive ruthenium chelate based monomer with the monomer linker having surfaces;
conjugating and cross-linking of a protein to micro or nanosurfaces by an initiator through the aminoacid-monomer linkages to give covalent bonds with tyrosine, cysteine or a tryptophane of antibodies and other biomolecules.

2. The method of claim 1, wherein the aminoacid or aminoacid monomers containing Ruthenium based metal-chelate complexes [Ru (bipyr)₂ L′L″] having the function of photo-linker-mediated conjugation.

3. The aminoacid monomer having the function of both a photosensitizer and linker according to claim 2, wherein the monomer is, 3-(4-hydroxyphenyl)-2-[(2 methylacryloyl)-amino]propanoic acid (methacryloyl tyrosine, MATyr) and/or N-[1-(1H-Indol-3-ylmethyl)-2-oxo-propyl]-2-methylacrylamide (methacryloyl tryptophan, MATrp), and/or 2-[(2-methylacryloyl)-amino]-3-sulfanylpropanoic acid (methacryloyl cysteine, MACys).

4. A method of making photosensitive bio-conjugation of antibodies and other proteins on surfaces, the ANADOLUCA (Aminoacid decorated and light underpining conjugation approach) method comprising the steps of:
preparing an acryloyl, methacryloyl or vinyl-monomer linker having surfaces;
preparing an aminoacid-monomer having surfaces;
making cross-linking conjugation of proteins to aminoacid monomer having micro and nanosurfaces by photosensitive oligomer and/or polymer or bis(2-2′-bipyridyl)ruthenium (II) solution mediated

5. The method of claim 4, wherein the amino acid monomer based modification way is selected from the group consisting of: MATyr, MATrp, and MACys and/or MUABt/aminoacid monomer.

6. The method of claim 4, wherein chemical synthesis of a vast range molecular weight having photosensitive oligomer of [Ru (bipyr)₂ using aminoacid monomer having Ruthenium metal chelates.

7. The method of claim 4, wherein the monomer linker having surface is selected from the group consisting of: nano-protein beads, iron-oxide particles, quantum dots, Au/Ag nanoclusters, silica nanoparticles, carbon nanotube (CNT), cryogel, Surface Plasmon Resonance (SPR) chips, Quartz Crystal Microbalance (QCM) electrodes, and organoclay.

8. The method of claim 7, wherein nano-powderized and functionalized protein is a polymeric material that has been prepared using a protein monomer and a MACys, MATyr and MATrp based aminoacid monomers or Ruthenium based aminoacid monomer hapten by light induced micro-emulsion polymerization.

9. The method of claim 8, wherein a composition comprising:
interacting the protein and Ruthenium based photosensitive aminoacid monomer hapten or synergie between aminoacid monomer and photosensitive oligomer and an initiator through micro-emulsion;
determining the activity of a nano protein bead.

10. The composition of claim 9, wherein the protein is a plasma protein (carrier or cell receptor ligand), an antibody, an enzyme, a biotin recognized protein or a lectin.

11. The composition of claim 10, wherein the plasma protein is an albumin (BSA), a transferrin or a TNF-α, the antibody is an Immunoglobulin G or antitubulin antibody, the enzyme is a lipase or a lysozyme, the lectin is a conconavalin A, the biotin recognized protein is a avidin.

12. A method of interaction between nano protein beads and its conjugates and nano heterobifunctional protein beads, the method comprising:
detecting and comprising the fluorescence intensity of nano protein beads and its conjugates;
determining the affinity interaction between nano protein beads and its recognition twin; and/or
interaction with cancer cells.

13. A method of preparing a nano protein or nano heterobifunctional protein according to claim 12, the method comprising the steps of:
preparing a microemulsion media using dispersed polyvinylalcohol (PVA) in deionized water;
preparing ruthenium based amino acid monomer hapten-protein or protein mixture complex structure or aminoacid monomer (one of MACys, MATyr, MATrp)-protein or protein mixture preorganisation through photosensitive ruthenium based hapten oligomer;
covalent conjugation and polymerization of protein or protein mixture complex in microemulsion media without protein denaturation.

14. The method according to claim 13, wherein the nano proteins are conjugated through coupling agents and/or via aminoacid monomer hapten through covalent cross-linking conjugation methods.

15. The method according to claim 14, wherein the BSA or heterobifunctional BSA nanocargoes are conjugated with one or more other molecules or proteins as a conjugated-multiplex protein system.

16. The method of claim 15, wherein said nano protein particles are labeled according to decorated ruthenium based aminoacid monomer hapten for fluorescence labeling.

17. A method of testing the efficiency of nanotransferrin that has been prepared according to claim 14, the method comprising the steps of:
affinity interaction between nanotransferrin particles and antitransferrin antibody
solution using fluorescence studies;
conjugation of anticancer drug ;
interaction with cancer cells by using flow cytometry.

18. A method of preparing and testing the efficiency of the tumor necrosis factor (TNF-α) nanoparticles (nanoTNF-α) according to claim 13, the method comprising the steps of:
preparing a microemulsion media using dispersed polyvinylalcohol in deionized water;
preparing aminoacid monomer hapten-TNF-α complex structure;
covalent conjugating and photosensitive polymerizating of TNF-α-monomer complex in microemulsion media;
interacting the nano TNF-α (cell receptor ligand) with cancer cells.

19. A method of preparing and analysing nano IgG cargo structure to carry the proteins, for antibacterial therapy and for using in antigen multipoint interaction according to claim 14, said method comprising:
preparing a microemulsion media using dispersed polyvinylalcohol in deionized water;
preparing a Ruthenium based amino acid monomer hapten-IgG complex structure;
covalent conjugation and polymerization of antibody complex in microemulsion media;
conjugating nano IgG to Gold-Protein A structure for providing active antigen binding sites of Nano IgG;
multipoint interaction of antibodies with nano IgG conjugated gold-Protein A;
analysing of cargo molecule activity and multipoint interaction activity by using fluorescence spectroscopy.

20. The method of claim 19, comprising four phases to prove multi binding sites of gold protein A which is originally found in the cell wall of the bacteria, Staphylococcus aureus, and it binds through the Fc region of immunoglobulins and especially showing binding activity of all attaching molecules with fluorescence analysis; wherein
a first phase is preparing the complex of gold-protein A and nano IgG and assaying the interaction;
a second phase is preparing anti IgG antibody interaction with nano IgG conjugated gold-protein A ([anti IgG-nano IgG] conjugated gold protein) and detecting the interaction with fluorescence spectroscopy;
a third phase is preparing anti IgM antibody interaction with conjugated gold-protein A and detecting the interaction with fluorescence spectroscopy;
a fourth phase is preparing IgM interaction with anti IgM antibody decorated and anti IgG antibody-nano IgG conjugated gold-protein A and detecting the interaction with fluorescence spectroscopy.

21. A method of preparing and assaying a lipase nano enzyme according to claim 13, the method comprising the steps of:
preparing a microemulsion media using dispersed polyvinylalcohol in deionized water;
preparing one of MACys, MATyr, or MATrp-lipase complex amino acid monomer structure;
covalent conjugation and polymerization of lipase complex in microemulsion media through aqueous solution of ruthenium based hapten polymer without enzyme denaturation;
contacting the nanolipase for hydrolysis of p-nitrophenyl palmitate where in the conversion is performed in the presence of nano-lipase enzyme;
determining reusability of the nanolipase.

22. A method of preparing and testing the efficiency of the lysozyme nanoparticle according to claim 13, the method comprising the steps of:
preparing a microemulsion media using dispersed polyvinylalcohol in deionized water;
preparing aminoacid monomer or hapten-lysozyme complex structure;
covalent conjugating and photosensitive polymerizing of the protein-monomer in microemulsion media;
testing the antibacterial efficiency of the nano lysozyme.

23. A method of preparing and assaying a concanavalin A nanoparticle according to claim 13, the method comprising the steps of:
preparing a microemulsion media using dispersed polyvinylalcohol in deionized water;
preparing Ruthenium based amino acid monomer hapten-Con A complex structure;
covalent conjugation and polymerization of Con A complex in microemulsion media;
assaying mannose and IgM using the nano Con A particles with fluorescence spectroscopy.

24. A method of preparing and biotin labeling a polymeric nano avidin according to claim 13, the method comprising the steps of:
preparing a microemulsion media using dispersed polyvinylalcohol in deionized water;
preparing Ruthenium based amino acid monomer hapten-avidin complex structure;
covalent conjugation and polymerization of avidin antibody complex in microemulsion media at daylight and room temperature;
interacting the nano avidin with biotin and biotinylated affinity groups;
assaying the affinity interaction of the nano avidin with fluorescence spectroscopy.

25. A method of preparing and tubulin labeling a polymeric antitubulin antibody according to claim 13, the method comprising the steps of:
preparing a microemulsion media using dispersed polyvinylalcohol in deionized water;
preparing Ruthenium based amino acid monomer hapten-antitubulin antibody complex structure;
covalent conjugation and polymerization of antitubulin antibody-monomer complex in microemulsion media at daylight and room temperature;
interacting the nano antitubulin with tubulin solution;
assaying the affinity interaction with fluorescence spectroscopy.

26. A method of preparing and testing of the protein conjugated superparamagnetic nanoparticle (SPN) according to claim 7, the method comprising the steps of:
SPN activating with 3-(Trimethoxysilyl)propyl methacrylate;
conjugating photosensitive aminoacid monomer of methacryloyl coated SPN;
conjugating and crosslinking the SPN without a protein denaturation;
targeting and affinity interaction the protein conjugated SPN nanomagnets as specific trap, cellular imager and nanotheranostics.

27. The method according to claim 26, the preparation of photosensitive protein conjugation of activated SPN for superoxide dismutase (SOD) and TNF-α separation, purification and affinity studies.

28. A method according to claim 26, the preparation of both transferrin and folic acid conjugated iron oxide nanoparticles using amino acid monomer and NHS-EDC coupling agents for killing human cancer cells as nanotheranostics.

29. A nanostructure according to claim 7, wherein the nanostructure is photosensitive monomer or hapten monomer linked to a hydrophilic quantum dot.

30. A method of preparing a protein conjugated hydrophilic CdS quantum dots according to claim 29, the method comprising the steps of:
quantum dot activating with hydrophilic thiol having aminoacid monomer or hapten monomer,
conjugating photosensitive aminoacid monomer of methacryloyl coated quantum dots;
binding and crosslinking the quantum dot without protein denaturation for antigen detection, cellular imaging, targeting and nanotheranostics.

31. The method according to claim 30, in the preparation of photosensitive antiTNT antibody, glycoprotein and anti-Hepatit B conjugation of monomer activated CdS nanocrystals for trinitrotoluen (TNT), IgM and Hepatit B detection, imaging, therapy and diagnosis and affinity studies.

32. A nanostructure according to claim 7, wherein the nanostructure is photosensitive monomer or hapten monomer linked to a gold nanoparticle or Ag/Au nanocluster.

33. A method of preparing an affinity pair cross-linked shell having Au/Ag nanocluster according to claim 32, the method comprising the steps of:
Au/Ag nanocluster activating with MACys or thiol having aminoacid monomer hapten;
conjugating photosensitive aminoacid monomer of methacryloyl coated Au/Ag nanocluster;
binding and crosslinking affinity pair (protein-template) onto Au/Ag nanocluster without protein unless denaturation for antigen detection, affinity studies, orientation and targeting and as nanotheranostics.

34. The method according to claim 33, the preparation of photosensitive avidin protein and its affinity pair polymerised using cross-linker and preparing imprinted shell of nanostructures using leaching, for affinity pair detection and separation.

35. The method according to claim 31 further comprising ambush and cell targeting of the following steps:
conjugating aminoacid monomer or hapten modified quantum dots with P450 reductase from rabbit liver;
decorating QD-P450 conjugate with transferrin targeting protein using photosensitive monomer coupling agent and covalent conjugation by ruthenium based polymer solution;
targeting HL-60 cancer cells with transferrin decorated QD-P450 reductase conjugate;
treating said cells with antitransferrin and prodrug decorated Au—Ag nanocluster;
avtivating prodrug on to Ag/Au nanocluster by the ambushed QD-P450 reductase conjugate;
killing said cancer cells.

36. A method of preparing a photosensitive protein conjugated cryogel according to claim 7, the method comprising the steps of:
preparing acrylamide, a photosensitive aminoacid monomer or hapten monomer and a protein mixture;
polymerizing of the monomer mixture in situ cryo and photo; or embedding the silica (or others) nanoparticles in the cavities of cryogel.

37. A method according to claim 36, the preparation of photosensitive cryogel-protein conjugation and cross-linking for using in bioseparation and as an antimicrobial material.

38. A method of preparing a photosensitive protein conjugated silica nanoparticles through doped ruthenium based polymer according to claim 37, the method comprising the steps of:
preparing silica nanoparticles doped with photosensitive ruthenium based aminoacid polymer;
methacryloyl-coating of doped silica nanoparticles;
conjugating methacryloyl coated particles with aminoacid monomer;
protein cross-linking onto silica nanoparticles through doped ruthenium based polymer for antigen detection, affinity studies, and imaging and as nanotheranostics.

39. A method of preparing a protein conjugated carbon nanotube according to claim 7, the method comprising the steps of:
a carbon nanotube oxidizing;
reacting MAArg monomer with the oxidized carbon nanotube to form methacryloyl coated nanostructure;
conjugating photosensitive amino-acid monomer of methacryloyl coated carbon nanotube;
binding and cross-linking the protein onto carbon nanotube without a protein unless denaturation for using in antigen detection, affinity and imaging studies.

40. A method for synthesizing CNT-fluorescent annexin V conjugate according to claim 39, that is photostable and can be used to detect apoptosis by fluorescence microscopy.

41. A method of preparing a protein cross-linked organoclay according to claims 7, the method comprising the steps of:
preparing a clay, comprising smectite;
modifyingmodificating of acrylamido-2-methyl-1-propansulfonicacid to the smectite for the monomeric modification in a nanolayer of organoclay;
aminoacid monomer linked ruthenium based hapten conjugation to the modified organoclay;
protein photo-entrapping and crosslinking within nanolayers of organoclay for reusable enzyme applications.

42. A method of preparing protein cross-linked and gold coated QCM and SPR sensors according to claim 7, the method comprising the step of:
cleaning the sensors with a piranha solution;
modifying using thiol containing photosensitive aminoacid monomer or ruthenium based hapten monomer;
conjugating the proteins on sensors surface using photopolymerization without denaturation for biosensor based applications.
